SEQUENCE LISTING (15 sequences)

SEQ ID NO: 1 - DNA, 21 bp, Artificial Sequence
  Human HGF forward
cagcgttgggattctcagtat

SEQ ID NO: 2 - DNA, 21 bp, Artificial Sequence
  Human HGF reverse
cctatgtttgttcgtgttgga

SEQ ID NO: 3 - DNA, 22 bp, Artificial Sequence
  Human MET forward
acagtggcatgtcaacatcgct

SEQ ID NO: 4 - DNA, 20 bp, Artificial Sequence
  Human MET reverse
gctcggtagtctacagattc

SEQ ID NO: 5 - DNA, 21 bp, Artificial Sequence
  Human Beta-actin forward
ggcggcaacaccatgtaccct

SEQ ID NO: 6 - DNA, 21 bp, Artificial Sequence
  Human Beta-actin reverse
aggggccggactcgtcatact

SEQ ID NO: 7 - DNA, 21 bp, Artificial Sequence
  Human EGFR forward
cttcttgcagcgatacactgc

SEQ ID NO: 8 - DNA, 21 bp, Artificial Sequence
  Human EGFR reverse
atgctccaataaattcactgc

SEQ ID NO: 9 - DNA, 18 bp, Artificial Sequence
  Human EGFRvIII forward
atgcgaccctccgggacg

SEQ ID NO: 10 - DNA, 22 bp, Artificial Sequence
  Human EGFRvIII reverse
attccgttacacactttgcggc

SEQ ID NO: 11 - DNA, 2576 bp, Homo sapiens
  GenBank Accession No. M29145  GI184041 (1)..(2576)
gggctcagagccgactggctcttttaggcactgactccgaacaggattctttcacccaggcatctcctccagagggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgaagtaagtgtgtctgaagcacccaccaatacaactgtcttttacatgaagatttcagagaatgtggaatttaaaatgtcacttacaacaatcctaagacaactactggagagtcatgtttgttgaaattctcattaatgtttatgggtgttttctgttgttttgtttgtcagtgttattttgtcaatgttgaagtgaattaaggtacatgcaagtgtaataacatatctcctgaagatacttgaatggattaaaaaaacacacaggtatatttgctggatgataa

SEQ ID NO: 12 - protein, 728 aa, Homo sapiens
  GenBank Accession No. AAA64239 GI337936 (1)..(728)
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 13 - protein, 95 aa, Homo sapiens
  MISC_FEATURE (1)..(95): Human MET IPT/TIG domain 3
PIVYEIHPTKSFISGGSTITGVGKNLNSVSVPRMVINVHEAGRNFTVACQHRSNSEIICCTTPSLQQLNLQLPLKTKAFFMLDGILSKYFDLIYV

SEQ ID NO: 14 - DNA, 18 bp, Artificial Sequence
  Human HGF Forward
ctccccatcgccatcccc

SEQ ID NO: 15 - DNA, 18 bp, Artificial Sequence
  Human HGF reverse
caccatggcctcggctgg

(19) United States
(12) Patent Application Publication
(10) Pub. No.: US 2012/0100157 A1
(43) Pub. Date: Apr. 26, 2012

(54) Biomarker and Method for Predicting Sensitivity to MET Inhibitors

(75) Inventors: George F. Vande Woude, Ada, MI (US); Qian Xie, Grand Rapids, MI (US)

(73) Assignee: Van Andel Research Institute, Grand Rapids, MI (US)

(21) Appl. No.: 13/270,686

(22) Filed: Oct. 11, 2011

Related U.S. Application Data
(60) Provisional application No. 61/391,806, filed on Oct. 11, 2010.

(51) Int. Cl. A61K 39/395; G01N 21/64; G01N 21/76; A61P 35/00; C12Q 1/68; C40B 30/00; C40B 30/04; G01N 33/566; G01N 33/82

(52) U.S. Cl. 424/174.1; 436/501; 435/7.9; 435/7.92; 435/7.1; 435/6.11; 506/7; 506/9

(57) ABSTRACT

Methods for determining the responsiveness of a Met-related cancer in a subject to treatment with a Met inhibitor. Kits for performing the disclosed methods are also provided. The present invention also provides a method of treating glioblastomamultiforme (GBM) in a subject in need thereof, the method comprises administering a therapeutically effective dose of a Met inhibitor in combination with a therapeutically effective dose of a epithelial growth factor receptor (EGFR) inhibitor.

### CROSS-REFERENCE TO PRIOR APPLICATIONS

This application claims the benefit of U.S. Provisional Application No. 61/391,806 filed Oct. 11, 2010, which is hereby incorporated by reference in its entirety.

### FIELD

The present invention relates to methods for determining the responsiveness of a met-related cancer to cancer therapeutics. In particular, the present invention relates to determining the responsiveness of met-related cancer to met-inhibitors. Methods and kits for performing such methods are also described.

### BACKGROUND

The statements in this section merely provide background information related to the present disclosure and may not constitute prior art.

Glioblastomamultiforme (GBM) is the most devastating brain cancer. The intrinsic capability of these tumor cells to invade normal brain impedes complete surgical eradication and predictably results in early local recurrence and mortality. Understanding the molecular mechanisms of GBM invasiveness will lead to novel therapeutic strategies applicable before and/or after surgical intervention and optimize the chances of preventing local recurrence.

The RTK c-Met is the cell surface receptor for hepatocyte growth factor (HGF), also known as scatter factor. HGF is a 90 kD multidomain glycoprotein that is highly related to members of the plasminogen serine protease family. It is secreted as a single-chain, inactive polypeptide by mesenchymal cells and is cleaved to its active α/β heterodimer extracellular form by a number of proteases. The α chain NH₂-terminal portion contains the high-affinity c-Met receptor-binding domain, but the β chain is required to interact with the c-Met receptor for receptor activation. The c-Met receptor, like its ligand, is a disulfide-linked heterodimer consisting of extracellular α and β chains. The α chain, heterodimerized to the amino-terminal portion of the 3 chain, forms the major ligand-binding site in the extracellular domain. The transmembrane domain, and the juxtamembrane region containing the receptor downmodulation c-Cbl-binding domain, is adjacent to the kinase domain and the carboxy-terminal tail that is essential for downstream signaling. HGF binding induces c-Met receptor homodimerization and phosphorylation of two tyrosine residues (Y1234 and Y1235) within the catalytic site, regulating kinase activity. The carboxy-terminal tail includes tyrosines Y1349 and Y1356, which, when phosphorylated, serve as docking sites for intracellular adaptor proteins, leading to downstream signaling.

The c-Met receptor is expressed in the epithelial cells of many organs during embryogenesis and in adulthood, including the liver, pancreas, prostate, kidney, muscle, and bone marrow. HGF mediated activation of the c-Met receptor tyrosine kinase (RTK) leads to tumor “invasive growth” (1-4). HGF binds to its receptor Met and triggers a series of intracellular signaling pathways leading to multiple activities, including cell proliferation, invasion, survival, and angiogenesis. The major signaling pathways driving tumor invasion and metastasis are RAS-MAPK and Akt, which are also the leading pathways that result in GBM tumorigenesis and invasion (5, 6). Aberrant Met activation can occur through binding of its ligand HGF following autocrine stimulation, paracrine stimulation, or ligand-independent activation resulting from gene amplification (1), transcriptional up-regulation (7), Met mutation (8) or cross-talk with other RTK family members. Met amplification is found to be the major secondary driver of tumor growth after acquired resistance to EGFR inhibitors (9).

Met overexpression is found in most glioblastomas and some glioblastomas display HGF-autocrine activation (10). Recent studies have shown that approximately 88% of GBM patients have an aberrant RTK/RAS-PI3K pathway. Met is located on Chromosome 7q. While gain of chr.7 frequently occurs in GBM patients, high level of Met amplification was found in approximately 4% of the patients (11, 12), Met mutation seems rare in brain cancer, although it is found in papillary renal cell tumors (13).

Amplification of EGFR occurs in 45% of GBM and is often associated with aberrant Met(11). Studies have shown that HGF can transcriptionally activate EGFR signaling in GBM cell lines (14). EGFR variant III (vIII) overexpression can activate Met signaling (15), raising the importance of using a combination of Met and EGFR inhibitors in targeting GBM. EGFRvIII and Met inhibitors synergize against PTEN-null/EGFRvIII+ GBM xenografts (16). Because both Met and EGFR inhibitors are being tested against GBM in clinical trials (17-19), it is increasingly important to identify mechanistic determinants that can predict drug sensitivity. Knowledge of the factors determining sensitivity or resistance to Met or EGFR inhibitors will improve the identification of patient subgroups suitable for Met and EGFR inhibition and will aid in the design of tailored combination strategies.

### SUMMARY

HGF binds to its receptor Met, leading to tumor invasive growth including glioblastoma. EGFR amplification frequently occurs in glioblastoma and is often associated with Met aberrancy. Because Met and EGFR inhibitors are in clinical development against several types of cancer including glioblastoma, it is important to identify accurately predictive determinants that indicate subject subgroups suitable for these specific therapies. The present inventors investigated in vivo glioblastoma models for their susceptibility to Met inhibitors sustained by either HGF-autocrine or HGF-paracrine activation or by Met and EGFR amplification. HGF-autocrine expression correlated with p-Met levels in HGF-autocrine cell lines, and show high sensitivity to Met inhibition in vivo. An HGF-paracrine environment could enhance glioblastoma growth in vivo but did not indicate sensitivity to Met inhibition. EGFRvIII amplification predicted sensitivity to EGFR inhibition, but amplified Met from gain of chromosome 7 in the same tumor did not display Met activity and did not predict sensitivity to Met inhibition. Thus, HGF-autocrine glioblastoma bears an activated Met signaling pathway that predicts their sensitivity to Met inhibitors in glioblastoma subjects. Moreover serum HGF levels may serve as a significant biomarker for the presence of autocrine tumors and their response to Met therapeutics, for example, Met inhibitors. The inventors establish a link between HGF-autocrine status and sensitivity to Met inhibition in cancers, including glioblastoma. If HGF-autocrine status is a biomarker, it can be used in clinical settings to rapidly identify subjects suitable for treatment with Met therapeutics.

In one aspect, the present invention provides a method for determining the responsiveness of a Met-related cancer in a subject to treatment with a Met inhibitor. The method includes, in a subject having or is suspected of having a Met-related cancer, the steps of detecting whether the Met-related cancer is HGF-autocrine; and if the Met-related cancer is HGF-autocrine, determining that the Met-related cancer will be responsive to treatment with a Met inhibitor.

In various embodiments of the methods described herein, the step of detecting whether the Met-related cancer is HGF-autocrine can include: (a) obtaining a biological sample from the subject; (b) measuring the level of expression of HGF in the biological sample; and (c) comparing the level of expression of HGF present in the biological sample to a reference sample. If the level of expression of HGF in the biological sample is different from the level of expression of HGF in the reference sample, the subject harbors a Met expressing HGF-autocrine tumor.

In another aspect, the step of obtaining a biological sample from the cancer subject can include: obtaining a blood sample from the subject, obtaining a tumor tissue specimen from the subject, obtaining a cerebral spinal fluid (CSF) sample from the subject, obtaining a tissue biopsy, obtaining a surgically resected tumor tissue sample or combinations thereof.

In various embodiments, the method step of measuring the level of expression of HGF in a biological sample can include contacting the biological sample with a HGF-binding reagent under suitable conditions (e.g. incubation conditions, pH, temperature, time etc) to promote the binding of HGF with the HGF-binding reagent. The HGF-binding reagent which finds utility in the present invention can include: an anti-HGF antibody or HGF-binding fragment thereof, a nucleic acid operable to hybridize to at least a portion of a HGF gene or transcription product thereof, a HGF binding protein, and combinations thereof.

In one aspect, the methods of the present invention utilize a comparison step in which the level of expression of HGF in the biological sample is greater than the level of expression of HGF in the reference sample.

In some embodiments, the HGF-binding agents are labeled with a detectable tag. Examples of detectable tags can include: a fluorophore, a radioligand, a chemilluminescence molecule, a conjugated enzyme, a peptide conjugate molecule, and combinations thereof. In one embodiment, the anti-HGF antibody is labeled or conjugated with a detectable tag, for example, a fluorophore, (fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) and N,N,N′,N′-tetramethyl-6-carboxyrhodamine (TAMRA)), a conjugated enzyme, (for example, horseradish peroxidase, or alkaline phosphatase), labeled with a radioisotope, (for example, radiolabeled methionine, iodine 131, ytterium 90 and phosphorus 32), or conjugated with a peptide conjugate molecule (for example, avidin, streptavidin, biotin, 6-8X-His, glutathione S-transferase (GST), and FLAG).

In another aspect, the methods of the present invention also include a comparison step. In this step, the level of expression of HGF present in the biological sample is compared to the expression level of HGF in a reference sample. If the level of expression of HGF in the biological sample is different from the level of expression of HGF in the reference sample, the subject harbors a Met expressing HGF-autocrine tumor. The comparison step can be qualitative, for example, visualization of the HGF-binding agent bound to HGF in the biological sample and/or quantitative, for example, the amount or concentration of HGF found in a subject's biological sample or in a reference sample is quantified and determined. If the subject is found to have a Met-related cancer, and the Met-related cancer is HGF-autocrine, then the subject's Met-related cancer will be responsive to treatment with a Met inhibitor.

In another aspect, the present invention includes a kit for determining the responsiveness of a tumor to Met inhibition. The kit can include a container for collecting a biological sample from a subject diagnosed with a Met-related cancer or a subject suspected of having a Met-related cancer, and a HGF-binding reagent for detecting HGF in the biological sample. In some embodiments, instructions may be provided to instruct an operator how to use the kit components.

In still another aspect, the present invention provides a method for treating glioblastomamultiforme (GBM) in a subject. The method includes administering a therapeutically effective dose of a Met inhibitor in combination with a therapeutically effective dose of an epithelial growth factor receptor (EGFR) inhibitor

Unless otherwise defined, all technical and scientific terms used herein have the same meaning as commonly understood by one of ordinary skill in the art to which this present technology belongs. Although methods and materials similar or equivalent to those described herein can be used in the practice or testing of the present technology, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the present invention are set forth in the accompanying figures and the description below. Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1A depicts a microarray heat map of general transcriptional changes between parental and M2 cell lines in vivo and in vitro.

FIG. 1B depicts an ingenuity pathway analysis (IPA) with in vivo data defining the HGF signaling pathway as one of the top 8 canonical pathways associated with cancer signaling/cell growth pathways with most genes upregulated (in Red).

FIG. 2A depicts a microarray heat map of HGF expression changes between parental and M2 cell lines in vivo and in vitro.

FIG. 2B depicts a photograph of a western blot of GBM cell lines and subclones showing that up-regulation of HGF expression is accompanied by increased p-MET.

FIG. 2C depicts a photograph of a western blot of U87M2 and DBM2 cells in response to HGF or EGF (100 ng/ml) stimulation and inhibition after 24 hours treatment.

FIG. 3 depicts a photograph of ethidium bromide stained gel showing expression levels of HGF, MET, EGFR, EGFRvIII, and β-actin in GBM cell lines, as determined by RT-PCR.

FIG. 4A depicts a graph of tumor growth representing SF295 cells (5×10⁵) inoculated subcutaneously into SCID and SCIDhgf mice. Tumors grow faster in SCIDhgf mice than in SCID mice.

FIG. 4B depicts depicts a graph of tumor growth representing SF295SQ1 cells isolated from a faster-growing SF295 tumor in SCIDhgf mouse. When inoculated in vivo again, there was no growth difference between tumors in SCID or SCIDhgf mice.

FIG. 5A depicts a bar chart representing inhibition of HGF- and EGF-induced uPA activity in U251M2 cell line incubated with 50 ng/ml HGF or EGF in the absence or presence of temozolomide (TMZ), erlonitib, or SGX523, as indicated. The uPA activity was determined after 24 hours after the treatment. Short bar represents for mean values+standard deviation from 4 replicates.

FIG. 5B depicts depicts a bar chart representing inhibition of HGF- and EGF-induced uPA activity in T98G cell line incubated with 50 ng/ml HGF or EGF in the absence or presence of temozolomide (TMZ), erlonitib, or SGX523, as indicated. The uPA activity was determined after 24 hours after the treatment. Short bar represents for mean values+standard deviation from 4 replicates.

FIG. 5C depicts depicts a bar chart representing inhibition of HGF- and EGF-induced uPA activity in DBM2 cell line incubated with 50 ng/ml HGF or EGF in the absence or presence of temozolomide (TMZ), erlonitib, or SGX523, as indicated. The uPA activity was determined after 24 hours after the treatment. Short bar represents for mean values+standard deviation from 4 replicates.

FIG. 5D depicts depicts a bar chart representing inhibition of HGF- and EGF-induced uPA activity in U87M2 cell line incubated with 50 ng/ml HGF or EGF in the absence or presence of temozolomide (TMZ), erlonitib, or SGX523, as indicated. The uPA activity was determined after 24 hours after the treatment. Short bar represents for mean values+standard deviation from 4 replicates.

FIG. 6A depicts growth curves of GBM cell line U87M2 in vivo in the presence and absence of paracrine human HGF expressed in SCID/hgf and SCID mice with and without a met inhibitor, a EGFR inhibitor and combination of met and EGFR inhibitors.

FIG. 6B depicts depicts growth curves of GBM cell line U118 in vivo in the presence and absence of paracrine human HGF expressed in SCID/hgf and SOD mice with and without a met inhibitor, a EGFR inhibitor and combination of met and EGFR inhibitors.

FIG. 6C depicts depicts growth curves of GBM cell line SF2955Q1 in vivo in the presence and absence of paracrine human HGF expressed in SCID/hgf and SCID mice with and without a met inhibitor, a EGFR inhibitor and combination of met and EGFR inhibitors.

FIG. 6D depicts depicts growth curves of GBM cell line U251M2 in vivo in the presence and absence of paracrine human HGF expressed in SCID/hgf and SOD mice with and without a met inhibitor, a EGFR inhibitor and combination of met and EGFR inhibitors.

FIG. 6E depicts depicts growth curves of GBM cell line DBMM2 in vivo in the presence and absence of paracrine human HGF expressed in SCID/hgf and SCID mice with and without a met inhibitor, a EGFR inhibitor and combination of met and EGFR inhibitors.

FIG. 7A depicts a growth chart of GBM cell line U87M2 in the presence of SGX523 (90 mg/kg) in combination with erlotinib (150 mg/kg).

FIG. 7B depicts depicts a growth chart of GBM cell line U87M2 in the presence of SGX523 (120 mg/kg) in combination with erlotinib (150 mg/kg).

FIG. 8A depicts a photomicrograph of a cytogenetic analysis of X01-GB stem cells in interphase (middle) and in metaphase (left and right). FISH signals detecting MET (red) and EGFR (green) show gain of MET and EGFR amplification as double minutes.

FIG. 8B depicts an ethidium bromide stained gel of results obtained by RT-PCR of MET, EGFR and EGFRvIII levels in X01-GB and V13 tumors

FIG. 8C depicts a photograph of a western blot of MET and EGFR expression and activation in X01-GB and V13 tumors.

FIG. 8D depicts a graph representing inhibition of growth of the GBM cell line X01-GB in the presence of f SGX523 and Erlotinib.

FIG. 8E depicts depicts a photomicrograph of a Cytogenetic analysis of V13 cells in metaphase, in primary tumor nuclei, and xenograft tumor nuclei show the same abnormalities. SKY analysis (upper left) shows trisomy 7 in the V13 cell line. FISH signals detecting MET (red), EGFR (green and aqua), HGF (green), and Chr.X (red) are indicated by the text color in the figure. Gain of Chr.7 is detected by SKY and metaphase FISH and a high level of EGFR amplification occurs as double minutes (upper panel).

FIG. 8F depicts depicts a graph representing inhibition of growth of the GBM cell line V13 in the presence of SGX523 and Erlotinib and combination thereof.

FIG. 9A depicts depicts a schematic representation of self organizing heat map based on transcriptional profiling of 202 GBM samples assayed by Cancer Genome Atlas research Network (2008) on Agilent 244K platform array. The map displays HGF, MET and EGFR transcripts (row) across the GBM samples (coloums). The dendrogram indicates the degree of similarity among GBM samples using Pearson's correlation coefficient. Genes were projected using log2 intensity and gene ratios were average corrected across experimental samples and displayed according to uncentered correlation algorithm. Red indicates over expression; green indicates under-expression; black indicates unchanged expression; gray indicates no detection of expression (intensity of both Cy3 and Cy5 below the cutoff value).

FIG. 9B depicts a schematic representation of a Matrix similarity based on Pearson's correlation for the HGF, MET and EGFR transcripts in GBM samples.

FIG. 9C depicts a table representing a CGH analysis displaying the frequency of amplifications occurring in A, B, C, D groups. P-value refers to the significance of correlation between EGFR, HGF and MET gene copy number alteration and their transcriptional levels described in FIG. 9A.

FIG. 9D depicts a Scatter plot between the average HGF intensity from the 4 groups (described in Table S4) and the percentage of samples determined with HGF-autocrine activation.

FIG. 9E depicts Scatter plot between the average MET in intensity of from 4 groups (described in Table 5) and the percentage of samples determined with HGF-autocrine activation.

FIG. 10A depicts a schematic representation of an in silico analysis of EGFR, HGF and MET genetic aberrancy in melanoma patients. Transcriptional profiling of 113 melanoma metastases; self organizing microarray heat map displaying HGF, MET and EGFR transcripts. Each column represents a melanoma metastasis.

FIG. 10B depicts a Matrix similarity based on Pearson's correlation for the HGF, MET and EGFR transcripts in GBM samples.

### DETAILED DESCRIPTION

The following description of technology is merely exemplary in nature of the subject matter, manufacture and use of one or more present inventions, and is not intended to limit the scope, application, or uses of any specific present technology claimed in this application or in such other applications as may be filed claiming priority to this application, or patents issuing therefrom. The following definitions and non-limiting guidelines must be considered in reviewing the description of the technology set forth herein.

The headings (such as “Background” and “Summary”) and sub-headings used herein are intended only for general organization of topics within the present technology, and are not intended to limit the disclosure of the present technology or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. Any discussion of the content of references cited in the present disclosure is intended merely to provide a general summary of assertions made by the authors of the references, and does not constitute an admission as to the accuracy of the content of such references. All references cited in the “Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present technology, or embodiments thereof, may alternatively be described using more limiting terms such as “consisting of” or “consisting essentially of” the recited ingredients.

Unless otherwise defined, all technical and scientific terms used herein have the same meaning as commonly understood by one of ordinary skill in the art to which this present technology belongs. Although methods and materials similar or equivalent to those described herein can be used in the practice or testing of the present technology, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

I. Methods for Predicting the Sensitivity of a Met-Related Cancer to Treatment with a Met Inhibitor

In some embodiments, the present invention provides a method for determining the responsiveness of a MET-related cancer in a subject to treatment with a Met inhibitor, the method includes the steps of: in a subject having or is suspected of having a Met-related cancer, detecting whether the MET-related cancer is HGF-autocrine; wherein if the Met-related cancer is HGF-autocrine, determining that the Met-related cancer will be responsive to treatment with a Met inhibitor.

As used herein, a “Met-related cancer” is defined as any cancer characterized by a cancer cell, tumor cell, or neoplastic cell that express a higher level of Met, either by gene amplification, protein expression or mRNA expression, as compared to non-cancer, non-tumor or non-neoplastic cells. Several cancer types are known to express higher levels of Met, for example, bladder, breast, cervical, cholangiocarcinoma, colorectal, endometrial, esophageal, gastric, head and neck, kidney, liver, lung, nasopharyngeal, ovarian, pancreas/gall bladder, prostate, thyroid, osteosarcoma, rhabdomyosarcoma, synovial sarcoma, Kaposi's sarcoma, leiomyosarcoma, MFH/fibrosarcoma, adult T-Cell leukemia, lymphomas, multiple myeloma, glioblastomas, (glioblastoma multiforme), melanoma, mesothelioma and Wilms tumor among others. In some embodiments, the Met-related cancer is glioblastoma multiforme.

In accordance with the present invention, the inventors have made the unexpected and surprising finding that Met-related cancers having an HGF-autocrine signaling loop are sensitive to Met inhibition. The HGF-autocrine signaling loop is therefore a biomarker for determining the sensitivity of a Met-related tumor cell to Met inhibition. As used herein, an HGF-autocrine loop for tumor activity exist in tumor cells that synthesize and secrete HGF, and become activated through the autocrine produced HGF, which also leads to tumorigenic responses to HGF. HGF-autocrine status therefore serves as a therapeutic marker for identifying subject subgroups for Met treatment. In some embodiments of the present invention, determining whether the subject with a Met-related cancer, or subject suspected of having a Met-related cancer, has an HGF-autocrine cancer includes detecting whether the Met-related cancer is HGF-autocrine. The present invention contemplates any method known in the art to determine whether the subject's Met-related cancer expresses HGF in an autocrine fashion.

Detecting whether a Met-related cancer expresses HGF in an autocrine fashion can include in vivo determination of HGF-autocrine status, for example, in vivo administration of radiolabeled antibodies directed to HGF, which are capable of binding to tumors expressing HGF in an autocrine fashion The HGF-autocrine labeled cancer can be subsequently detected by Computer Tomography (CT) scanning, Magnetic Resonance Imaging (MRI), Immuno-Positron Emission Tomography (iPET) scanning (Clin Cancer Res 12:1958-1960, 2006; Clin Cancer Res 12:2133-2140, 2006), and the like. Collectively, these imaging techniques are referred to in the art as radioimmunodetection of cancer, or radioimmunoscintigraphy. Approved radioisotopes for coupling with antibodies (chimeric, humanized, or antigen-binding fragments thereof) directed to cancer antigens or expression products for in-vivo use are known in the art. For in vivo detection of Met-related HGF-autocrine tumors, anti-HGF antibodies, or HGF-binding fragments thereof (Fab or F(ab′)₂, or ScFv) of the invention may be conjugated to radionuclides either directly or by using an intermediary functional group. An intermediary group which is often used to bind radioisotopes, which exist as metallic cations, to antibodies is diethylenetriaminepentaacetic acid (DTPA) or tetraaza-cyclododecane-tetraacetic acid (DOTA). Typical examples of metallic cations which are bound in this manner are ⁹⁹Tc ¹²³I, ¹¹¹In, ¹³¹I, ⁹⁷Ru, ⁶⁷Cu, ⁶⁷Ga, and ⁶⁸Ga. Moreover, the antibodies of the invention may be tagged with an Nuclear Magnetic Resonance (NMR) imaging agent which include paramagnetic atoms. The use of an NMR imaging agent allows the in vivo detection, diagnosis and presence of and the extent of HGF-autocrine tumor development and metastases in a subject using NMR techniques. Elements which are particularly useful in this manner are ¹⁵⁷Gd, ⁵⁵Mn, ¹⁶²Dy, ⁵²Cr, and ⁵⁶Fe. In a non-limited example, approved radioligands useful for in-vivo radioimmunodetection of HGF-autocrine cancer include ¹¹¹In and ⁹⁹Tc.

In some embodiments, detecting whether the Met-related cancer is HGF-autocrine can include the step of obtaining a biological sample from the subject. In such embodiments, detecting whether a Met-related cancer is HGF-autocrine includes the steps of: (a) obtaining a biological sample from the subject; (b) measuring the level of expression of HGF in the biological sample; and (c) comparing the level of expression of HGF present in the biological sample to a reference sample. If the level of expression of HGF in the biological sample is different from the level of expression of HGF in the reference sample, the Met-related cancer is HGF-autocrine (i.e., the subject harbors a Met expressing HGF-autocrine tumor) and the cancer will be responsive to treatment with a Met inhibiting agent. In some embodiments, if the level of expression of HGF in the biological sample is greater than the level of expression in the reference sample, the Met-related cancer is HGF-autocrine (i.e., the subject harbors a Met expressing HGF-autocrine tumor) and the cancer will be responsive to treatment with a Met inhibiting agent.

A. Biological Sample

In various embodiments of the present invention, the step of detecting whether the Met-related cancer is HGF-autocrine method step includes obtaining a biological sample from the subject diagnosed with a Met-related cancer or a subject suspected of having a Met-related cancer. In some embodiments, the biological sample can include a blood sample from the subject, a tumor tissue specimen from the subject, a cerebral spinal fluid (CSF) sample from the subject, or combinations thereof. A biological sample comprising a blood sample, can also include a serum sample, a plasma sample or whole blood sample from the subject. In one embodiment, the blood sample is a serum sample. As is customary in various FDA approved diagnostic procedures, the blood sample is maintained sterile and should be obtained using sterile equipment and approved phlebotomy techniques. Blood samples can range from 0.5 mL to approximately 50 mL, provided there is sufficient sample to detect the presence of an HGF-autocrine Met-related cancer, or a product expressed by an HGF-autocrine Met-related cancer, for example, HGF in the blood sample.

In another embodiment, obtaining a biological sample from the subject can include obtaining a tumor tissue specimen from the subject. Any means of sampling a tissue specimen from a subject, for example, by a tissue smear or scrape, or tissue biopsy can be used to obtain a sample. Thus, the biological sample can be a biopsy specimen (e.g., tumor, polyp, mass (solid, cell)), aspirate, or smear. The sample can be from a tissue that has a tumor (e.g., cancerous growth) and/or tumor cells, or is suspecting of having a tumor and/or tumor cells. For example, a tumor biopsy can be obtained in an open biopsy, a procedure in which an entire (excisional biopsy) or partial (incisional biopsy) mass is removed from a target area. Alternatively, a tumor sample can be obtained through a percutaneous biopsy, a procedure performed with a needle-like instrument through a small incision or puncture (with or without the aid of a imaging device) to obtain individual cells or clusters of cells (e.g., a fine needle aspiration (FNA)) or a core or fragment of tissues (core biopsy). The biopsy samples can be examined cytologically (e.g., smear), histologically (e.g., frozen or paraffin section) or using any other suitable method (e.g., molecular diagnostic methods). A biological sample can be obtained during a surgical procedure to excise or remove a tumor tissue sample in a subject, wherein the biological sample can be derived from the excised tumor mass, or by in vitro harvest of cultured human cells derived from an individual's suspected or confirmed Met-related cancer tissue excised during surgery, or biopsy.

For obtaining a biological sample of cultured cells isolated from a subject's cancer sample, >100 mg of non-necrotic, non-contaminated tissue can harvested from the subject by any suitable biopsy or surgical procedure known in the art. Biopsy sample preparation can generally proceed under sterile conditions, for example, under a Laminar Flow Hood which should be turned on at least 20 minutes before use. Reagent grade ethanol is used to wipe down the surface of the hood prior to beginning the sample preparation. The tumor is then removed, under sterile conditions, from the shipping container and is minced with sterile scissors. If the specimen arrives already minced, the individual tumor pieces should be divided into groups. Using sterile forceps, each undivided tissue section is then placed in 3 ml sterile growth medium (Standard F-10 medium containing 17% calf serum and a standard amount of Penicillin and Streptomycin) and systematically minced by using two sterile scalpels in a scissor-like motion, or mechanically equivalent manual or automated opposing incisor blades. This cross-cutting motion is important because the technique creates smooth cut edges on the resulting tumor multicellular particulates. Preferably but not necessarily, the tumor particulates each measure approximately 1 mm³. After each tumor quarter has been minced, the particles are plated in culture flasks using sterile pasteur pipettes (9 explants per T-25 or 20 particulates per T-75 flask). Each flask is then labeled with the patient's code, the date of explantation and any other distinguishing data.

The explants can be evenly distributed across the bottom surface of the flask, with initial inverted incubation in a 37° C. incubator for 5-10 minutes, followed by addition of about 5-10 mL sterile growth medium and further incubation in the normal, non-inverted position. Flasks are placed in a 35° C., non-CO₂ incubator. Flasks should be checked daily for growth and contamination. Over a period of a few weeks, with weekly removal and replacement of 5 ml of growth medium, the explants will foster growth of cells into a monolayer. With respect to the culturing of tumor cells, (without wishing to be bound by any particular theory) maintaining the malignant cells within a multicellular particulate of the originating tissue, growth of the tumor cells themselves is facilitated versus the overgrowth of fibroblasts (or other unwanted cells) which tends to occur when suspended tumor cells are grown in culture.

Tumor samples can, if desired, be stored before analysis by suitable storage means that preserve a sample's protein and/or nucleic acid in an analyzable condition, such as quick freezing, or a controlled freezing regime. If desired, freezing can be performed in the presence of a cryoprotectant, for example, dimethyl sulfoxide (DMSO), glycerol, or propanediol-sucrose. Tumor samples can be pooled, as appropriate, before or after storage for purposes of analysis.

In some embodiments, obtaining a biological sample from the subject can include obtaining a cerebrospinal fluid (CSF) sample using techniques known in the art. In one exemplary embodiment, a subject diagnosed with or suspected of having a Met-related cancer, for example a glioblastoma, is positioned with the back curved out so the spaces between the vertebrae are as wide as possible. This allows the medical practitioner to easily find the spaces between the lower lumbar bones (where the needle will be inserted). The medical practitioner carefully inserts a thin needle between the bones of the lower spine (below the spinal cord) to withdraw the fluid sample. The CSF fluid can then be frozen or processed to measuring the level of expression of HGF in the CSF sample.

B. Measuring HGF

In some embodiments, the methods provide a step which includes measuring the level of expression of HGF in the subject's biological sample. The method includes: contacting the subject's biological sample with a HGF-binding reagent under conditions to promote the binding of HGF with the HGF-binding reagent. In some embodiments, the HGF-binding reagent can be any agent that specifically binds to HGF protein or fragment thereof, or any nucleic acid operable to bind or hybridize with a nucleic acid that encodes HGF (for example, DNA, cDNA, RNA or mRNA). HGF-binding agents can include, without limitation: an anti-HGF antibody or HGF-binding fragment thereof, a nucleic acid operable to hybridize to at least a portion of a HGF gene, or RNA or cDNA transcript thereof, a HGF binding protein, and combinations thereof. Suitable assays can be used to assess the presence or amount of HGF in a sample (i.e., a biological sample). Methods to detect HGF can include immunological and immunochemical methods like flow cytometry (e.g., FACS analysis), enzyme-linked immunosorbent assays (ELISA), including chemiluminescence assays, radioimmunoassay, immunoblot (e.g., Western blot), and immunohistology/immunohistochemical methods, or other suitable methods such as mass spectroscopy. For example, antibodies to HGF can be used to determine the presence and/or expression level of HGF in a sample directly or indirectly using, for instance, immunohistology and/or immunohistochemistry. For instance, paraffin sections can be taken from a biopsy, fixed to a slide and combined with one or more labeled and/or unlabeled antibodies by suitable methods. Methods for immunogical detection of specific antigens in biological samples are known in the art.

Methods for detecting a HGF gene or expression product thereof (e.g., cDNA, RNA or mRNA) in a Met-related cancer can include visualizing, identifying and/or quantifying the level of expression of the HGF nucleic acid or expression product thereof. As used herein, “oligonucleotide” or “oligonucleotide probes” or “polynucleotide” or “nucleotide” or “nucleic acid” refer to a biological polymer molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

“Oligonucleotide having a nucleotide sequence encoding a gene” or “a nucleic acid sequence encoding” a specified polypeptide refer to a nucleic acid sequence comprising the coding region of a gene or in other words the nucleic acid sequence which encodes a gene product. The coding region may be present in either a cDNA, genomic DNA or RNA form. When present in a DNA form, the oligonucleotide may be single-stranded (i.e., the sense strand) or double-stranded. Suitable expression control sequences or elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

To detect a HGF gene or expression product thereof, a nucleic acid can be isolated from a subject's biological sample by suitable methods which are routine in the art (see, e.g., Sambrook et al., 1989). The isolated nucleic acid can then be amplified (by e.g., polymerase chain reaction (PCR) (e.g., direct PCR, quantitative real time PCR, reverse transcriptase PCR), ligase chain reaction, self sustained sequence replication, transcriptional amplification system, Q-Beta Replicase, or the like) and visualized (by e.g., labeling of the nucleic acid during amplification, exposure to intercalating compounds/dyes, probes). HGF gene or expression product thereof can also be detected using a nucleic acid probe, for example, a labeled nucleic acid probe (e.g., fluorescence in situ hybridization (FISH)) directly in a paraffin section of a tissue sample taken from, e.g., a Met-related tumor biopsy, or using other suitable methods. HGF gene or expression thereof can also be assessed by Southern blot or in solution (e.g., dyes, probes). Further, a gene chip, microarray, probe (e.g., quantum dots) or other such device (e.g., sensor, nanonsensor/detector) can be used to detect expression and/or differential expression of a HGF gene.

In one embodiment, HGF-binding reagents can include oligonucleotide probes that are operable to bind or hybridize under low, medium or high stringency, preferably high stringency to DNA or RNA nucleic acids that encode HGF. In some embodiments, biological samples that contain Met-related cancer cells that have intact DNA may be used to detect HGF-autocrine cancer cells. DNA and/or RNA can be extracted from such biological examples and probed with HGF specific oligonucleotide probes that are designed to specifically identify the presence of a HGF gene or portions thereof. The HGF specific oligonucleotide probes can be used in various detection assays to identify HGF nucleic acid expression. In one detection assay, oligonucleotide probes are incubated with DNA or cDNA obtained directly or indirectly from Met-related cancer samples and amplified using PCR. The amplification products can be sequenced to verify proper identification of the HGF gene or expression product thereof. (Barbi, S. et al. J. Experimental and Clinical Cancer Research 2010, 29:32) The assay could also be performed by only amplifying the tumor DNA and comparing the sequence with the sequence of SEQ ID NO:1.

In some embodiments, the present invention provides polynucleotide sequences comprising polynucleotide sequences in whole or in part from SEQ ID NO: 11 (GenBank Accession No. M29145 GI: 184041) or complementary sequences thereof, that are capable of hybridizing to the HGF gene under conditions of high stringency. Also contemplated, are HGF genes which are encoded by variant DNA and RNA transcripts, such as variant mRNA HGF transcripts and complementary sequences thereof known in the art. In some embodiments, the polynucleotides can include sequences complementary to nucleic acid sequences that encode in whole or in part HGF, for example, human HGF are contemplated herein. The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

In some embodiments, the present invention provides HGF-binding reagents useful in the detection of HGF-autocrine cancers. In one embodiment, polynucleotide sequences comprising polynucleotide sequences in whole or in part from SEQ ID NO: 11 or complementary sequences thereof, that are capable of hybridizing to the human HGF gene or portion thereof under conditions of high stringency find utility in the present invention. Utilizing the nucleotide databases publicly available (e.g. NCBI and GenBank), other polynucleotides or oligonucleotides can be synthesized to enable identification of HGF genes or expression products thereof from other species, for example, primate, and other mammals, for example, mouse, rat, rabbit, guinea pig, dos, cat and the like. In some embodiments, the polynucleotides can include sequences complementary to nucleic acid sequences that encode in whole or in part the HGF gene as described herein.

“High stringency conditions” when used in reference to nucleic acid hybridization comprise conditions equivalent to binding or hybridization at 42 C.° in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/mL denatured salmon sperm DNA followed by washing in a solution comprising 0.1×SSPE, 1.0% SDS at 42 C.° when a probe of about 500 nucleotides in length is employed.

The term “homology” when used in relation to nucleic acids refers to a degree of complementarity. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence which is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

In preferred embodiments, hybridization conditions are based on the melting temperature (Tm) of the nucleic acid binding complex and confer a defined “stringency” The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

The term “Tm” refers to the “melting temperature” of a nucleic acid. The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41 (% G+C), when a nucleic acid is in aqueous solution at 1 M NaCl. The term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences.

In some embodiments of the present invention, nucleotide sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the HGF gene) is sequenced using any suitable method, including but not limited to manual sequencing using radioactive marker nucleotides, or automated sequencing. The results of the sequencing are displayed using any suitable method. Methods for performing PCR are known in the art (see Current Protocols in Molecular Biology, edited by Fred M. Ausubel, Roger Brent, Robert E. Kingston, David D. Moore, J. G. Seidman, John A. Smith, Kevin Struhl. and; Molecular Cloning: A Laboratory Manual, Joe Sambrook, David W Russel, 3^{rd} edition, Cold Spring Harbor Laboratory Press).

The presence or absence of a Met-related HGF-autocrine cancer can be ascertained by the methods described herein or other suitable assays. In some embodiments, two or more methods for determining the presence of HGF-autocrine Met-related cancer cells can be used, for example a first detection method using nucleic-acid probes and a second method using anti-HGF antibodies to confirm the presence of HGF-autocrine cancer cells.

A determination whether a biological sample is HGF-autocrine can be made by comparison of the level of expression of HGF in the biological sample to that of a suitable control or reference sample. Suitable controls or reference samples include, for instance, a non-neoplastic tissue sample from the individual, non-cancerous cells, non-metastatic cancer cells, non-malignant (benign) cells or the like, cancer cell lines that are not HGF-autocrine, for example GBM cell lines DBM2 or U251M2 or a suitable known or determined standard. The reference sample can also include tumor cells from the patient upon diagnosis of the cancer, or before, during or after surgery as the tumor is being removed, or alternatively, a tumor sample taken before commencement of a cancer therapeutic regime (chemotherapeutics or radiation therapy). The reference sample can be a known or determined typical, normal or normalized range or level of expression of HGF protein or gene (e.g., an expression standard) for a non-HGF-autocrine signaling tumor or healthy cell. Therefore, the method does not require that expression of the gene/protein be assessed in a suitable control. HGF expression in the biological and/or reference sample from the subject known or suspected of having a Met-related cancer can be compared to its expression in known or determined standard. In one embodiment, the amount or quantification of HGF in the biological sample and/or reference sample can be determined using mathematical operations on a computer, or a CPU containing device. The CPU containing device can have both a memory component to store the data gathered on the level of expression of HGF in a subject's biological sample, and a memory component to store computer software to perform the mathematical calculations from the inputted data representing the level of HGF expression (for example, the amount or concentration of HGF) in a subject's biological and/or a reference sample. The calculated amounts of HGF in the biological sample and reference sample can be used to determine whether the Met-related cancer is HGF-autocrine.

The reference levels of HGF are related to the values used to characterize the level of HGF in the biological sample obtained from the subject. Thus, if the HGF level is an absolute value, then the reference value is also based upon an absolute value.

The reference levels can take a variety of forms. For example, a reference level of HGF can be a single cut-off value, such as a median or mean. Or, a reference level can be divided equally (or unequally) into groups, such as low, medium, and high groups, the low group being individuals least likely to be responsive to a Met-inhibitor and the high group being individuals most likely to be responsive to a Met-inhibitor.

Reference levels of HGF, e.g., mean levels, median levels, or “cut-off” levels, may be established by assaying a large sample of individuals in the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference.

The HGF levels in the biological sample may be compared to single control values or to ranges of control values. In one embodiment, an HGF level in a biological sample from a subject (e.g., a patient having or suspected of having a Met-related cancer) is present at a higher or lower level (i.e., at a different level) than in comparable reference biological samples when the HGF level in the biological sample exceeds a threshold of one and one-half standard deviations above the mean of the concentration as compared to the comparable reference biological samples. More preferably, an HGF level in a biological sample from a subject (e.g., a patient having or suspected of having a Met-related cancer) is present at a higher or lower level (i.e., at a different level) than in comparable reference biological samples when the HGF level in the biological sample exceeds a threshold of two standard deviations above the mean of the concentration as compared to the comparable reference biological samples. Most preferably, an HGF level in a biological sample from a subject (e.g., a patient having or suspected of having a Met-related cancer) is present at a higher or lower level (i.e., at a different level) than in comparable reference biological samples when the HGF level in the biological sample exceeds a threshold of three standard deviations above the mean of the concentration as compared to the comparable reference biological samples.

If the HGF level in the biological sample is present at a different level than the reference sample, then the subject is more likely to have a Met-related cancer that will be responsive to a Met inhibitor than are subjects with HGF levels comparable to the reference sample.

C. HGF-Binding Reagents

In some embodiments, the HGF-binding reagent can be any agent that specifically binds to HGF protein or fragment thereof, a nucleic acid operable to bind or hybridize with a nucleic acid that encodes HGF (for example, DNA, cDNA, RNA or mRNA), or a protein that binds to HGF (non-antibody). HGF-binding agents can include, without limitation: an anti-HGF antibody or HGF-binding fragment thereof, a nucleic acid operable to hybridize to at least a portion of a HGF gene, or RNA, or cDNA transcript thereof, a HGF binding protein, and combinations thereof. Suitable assays can be used to assess the presence or amount of HGF in a sample (i.e., a biological sample) using one or more of these HGF-binding reagents using assay methods described herein.

(i) HGF Antibodies

In some embodiments, HGF-binding agents include antibodies or HGF-binding fragments thereof. In some embodiments, an antibody that binds (e.g., specifically binds) to a HGF protein (e.g., a human HGF protein; SEQ ID NO:12), or a fragment thereof, are contemplated as HGF-binding reagents. Antibodies that specifically bind to a HGF protein or fragment thereof can be polyclonal, monoclonal, human, chimeric, humanized, primatized, veneered, and single chain antibodies, as well as fragments of antibodies (e.g., Fv, Fc, Fd, Fab, Fab′, F(ab′), scFv, scFab, dAb), among others. (See e.g., Harlow et al., Antibodies A Laboratory Manual, Cold Spring Harbor Laboratory, 1988). The term “antibody” includes, any immunoglobulin molecule that recognizes and specifically binds to a target, such as a protein, polypeptide, peptide, carbohydrate, polynucleotide, lipid, etc., through at least one antigen recognition site within the variable region of the immunoglobulin molecule. As used herein, the term is used in the broadest sense and encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes and the like.

In certain embodiments, the antibodies to HGF useful as HGF-binding reagents of the present invention have a high binding affinity for HGF, for example, human HGF. Such antibodies will preferably have an affinity (e.g., binding affinity) for HGF, of at least about 10⁻¹ M (e.g., about 0.5×10⁻⁷ M, about 0.5×10⁻⁷ M, or higher, for example, at least about 10⁻⁸ M, at least about 10⁻⁹ M, or at least about 10⁻¹⁰ M. The binding affinity of an antibody can be readily determined by one of ordinary skill in the art, for example, by Scatchard analysis (Scatchard, G., Ann. NY Acad. Sci. 51: 660-672, 1949). Binding affinity can also be determined using a commercially available biosensor instrument (BIACORE, Pharmacia Biosensor, Piscataway, N.J.), wherein protein is immobilized onto the surface of a receptor chip. See, Karlsson, J. Immunol. Methods 145:229-240, 1991 and Cunningham and Wells, J. Mol. Biol. 234:554-563, 1993. This system allows the determination of on- and off-rates, from which binding affinity can be calculated, and assessment of stoichiometry of binding.

“Antibody fragment” can refer to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments.

“Chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc) with the desired specificity, affinity, and capability while the constant regions are homologous to the sequences in antibodies derived from another (usually human) to avoid eliciting an immune response in that species.

“Humanized” forms of non-human (e.g., rabbit) antibodies include chimeric antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues that are not found in the recipient antibody or in the donor antibody. Most often, the humanized antibody can comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody can also comprise at least a portion of an immunoglobulin constant region (Fc), typically that of a human immunoglobulin. Methods used to generate humanized antibodies are well known in the field of immunology and molecular biology.

“Hybrid antibodies” can include immunoglobulin molecules in which pairs of heavy and light chains from antibodies with different antigenic determinant regions are assembled together so that two different epitopes or two different antigens can be recognized and bound by the resulting tetramer.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3-5, and more usually, at least 5 or 8-10 amino acids in a unique spatial conformation.

“Specifically binds” to or shows “specific binding” towards an epitope means that the antibody reacts or associates more frequently, and/or more rapidly, and/or greater duration, and/or with greater affinity with the epitope than with alternative substances.

Generally, procedures for production and use of antibodies, for example, immunoprecipitation, ELISA, and other uses of antibodies and related immunology methods and the like are common methods used in the art. Such standard techniques can be found in reference manuals such as for example, Kohler & Milstein (1975) Nature 256:495-497; Kozbor, et al. (1983) Immunology Today 4:72; Cole, et al., pp. 77-96 in Monoclonal Antibodies and Cancer Therapy (1985); Coligan (1991) Current Protocols in Immunology; Harlow & Lane (1988) Antibodies: A Laboratory Manual; and Goding (1986) Monoclonal Antibodies: Principles and Practice (2d ed.) all of these documents are incorporated herein in their entireties.

(a) Preparation of Antibodies

Polyclonal Antibodies

Polyclonal antibodies are preferably raised in animals by multiple subcutaneous (sc) or intraperitoneal (ip) injections of the relevant antigen and an adjuvant. Alternatively, antigen may be injected directly into the animal's lymph node (see Kilpatrick et al., Hybridoma, 16:381-389, 1997). An improved antibody response may be obtained by conjugating the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride or other agents known in the art.

Animals are immunized against the HGF protein, fragments thereof, immunogenic conjugates or derivatives thereof by combining, e.g., 100 μg of the protein or conjugate (for mice) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. At 7-14 days post-booster injection, the animals are bled and the serum is assayed for antibody titer. Animals are boosted until the titer plateaus. Preferably, the animal is boosted with the conjugate of the same antigen, but conjugated through a different cross-linking reagent. Conjugates also can be made in recombinant cell culture as protein fusions. Also, aggregating agents such as alum are suitably used to enhance the immune response.

Monoclonal Antibodies

Monoclonal antibodies can be made using the hybridoma method first described by Kohler et al., Nature, 256:495 (1975), or by recombinant DNA methods. In the hybridoma method, a mouse or other appropriate host animal, such as rats, hamster or macaque monkey, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells and are sensitive to a medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Exemplary murine myeloma lines include those derived from MOP-21 and M. C.-11 mouse tumors available from the Salk Institute Cell Distribution Center, San Diego, Calif. USA, and SP-2 or X63-Ag8-653 cells available from the American Type Culture Collection, Rockville, Md. USA. Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can be determined, for example, by BIAcore or Scatchard analysis (Munson et al., Anal. Biochem., 107:220 (1980)).

After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59-103 (Academic Press, 1986)). Suitable culture media for this purpose include, for example, D-MEMO or RPMI 1640 medium. In addition, the hybridoma cells can be grown in vivo as ascites tumors in an animal. The monoclonal antibodies secreted by the subclones are suitably separated from the culture medium, ascites fluid, or serum by conventional immunoglobulin purification procedures such as protein A-Sepharose, hydroxylapatite chromatography, gel electrophoresis, dialysis, or affinity chromatography.

Recombinant Production of Antibodies

The amino acid sequence of an immunoglobulin of interest can be determined by direct protein sequencing, and suitable encoding nucleotide sequences can be designed according to a universal codon table.

Alternatively, DNA encoding the monoclonal antibodies can be isolated and sequenced from the hybridoma cells using conventional procedures (e.g., by using oligonucleotide probes that are capable of binding specifically to genes encoding the heavy and light chains of the monoclonal antibodies). Sequence determination will generally require isolation of at least a portion of the gene or cDNA of interest. Usually this requires cloning the DNA or mRNA encoding the monoclonal antibodies. Cloning is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library can be constructed by reverse transcription of polyA+ mRNA, preferably membrane-associated mRNA, and the library screened using probes specific for human immunoglobulin polypeptide gene sequences. In a preferred embodiment, the polymerase chain reaction (PCR) is used to amplify cDNAs (or portions of full-length cDNAs) encoding an immunoglobulin gene segment of interest (e.g., a light chain variable segment). The amplified sequences can be cloned readily into any suitable vector, e.g., expression vectors, minigene vectors, or phage display vectors. It will be appreciated that the particular method of cloning used is not critical, so long as it is possible to determine the sequence of some portion of the immunoglobulin polypeptide of interest.

One source for RNA used for cloning and sequencing is a hybridoma produced by obtaining a B cell from the transgenic mouse and fusing the B cell to an immortal cell. An advantage of using hybridomas is that they can be easily screened, and a hybridoma that produces a human monoclonal antibody of interest selected. Alternatively, RNA can be isolated from B cells (or whole spleen) of the immunized animal. When sources other than hybridomas are used, it may be desirable to screen for sequences encoding immunoglobulins or immunoglobulin polypeptides with specific binding characteristics. One method for such screening is the use of phage display technology. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference. In one embodiment using phage display technology, cDNA from an immunized transgenic mouse (e.g., total spleen cDNA) is isolated, PCR is used to amplify cDNA sequences that encode a portion of an immunoglobulin polypeptide, e.g., CDR regions, and the amplified sequences are inserted into a phage vector. cDNAs encoding peptides of interest, e.g., variable region peptides with desired binding characteristics, are identified by standard techniques such as panning. The sequence of the amplified or cloned nucleic acid is then determined. Typically the sequence encoding an entire variable region of the immunoglobulin polypeptide is determined, however, sometimes only a portion of a variable region need be sequenced, for example, the CDR-encoding portion. Typically the sequenced portion will be at least 30 bases in length, and more often bases coding for at least about one-third or at least about one-half of the length of the variable region will be sequenced. Sequencing can be carried out on clones isolated from a cDNA library or, when PCR is used, after subcloning the amplified sequence or by direct PCR sequencing of the amplified segment. Sequencing is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of human immunoglobulin genes and cDNAs, an artisan can determine readily, depending on the region sequenced, (i) the germline segment usage of the hybridoma immunoglobulin polypeptide (including the isotype of the heavy chain) and (ii) the sequence of the heavy and light chain variable regions, including sequences resulting from N-region addition and the process of somatic mutation. One source of immunoglobulin gene sequence information is the National Center for Biotechnology Information, National Library of Medicine, National Institutes of Health, Bethesda, Md.

Once isolated, the DNA may be operably linked to expression control sequences or placed into expression vectors, which are then transfected into host cells such as E. coli cells, simian COS cells, Chinese hamster ovary (CHO) cells, or myeloma cells that do not otherwise produce immunoglobulin protein, to direct the synthesis of monoclonal antibodies in the recombinant host cells. Expression control sequences denote DNA sequences necessary for the expression of an operably linked coding sequence in a particular host organism. The control sequences that are suitable for prokaryotes, for example, include a promoter, optionally an operator sequence, and a ribosome-binding site. Eukaryotic cells are known to utilize promoters, polyadenylation signals, and enhancers.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome-binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking can be accomplished by ligation at convenient restriction sites. If such sites do not exist, synthetic oligonucleotide adaptors or linkers can be used in accordance with conventional practice.

“Cell line” and “cell culture” are often used interchangeably and all such designations include progeny. Transformants and transformed cells include the primary subject cell and cultures derived therefrom without regard for the number of transfers. It also is understood that all progeny may not be precisely identical in DNA content, due to deliberate or inadvertent mutations. Mutant progeny that have the same function or biological activity as screened for in the originally transformed cell are included.

Isolated nucleic acids also are provided that encode specific antibodies, optionally operably linked to control sequences recognized by a host cell, vectors and host cells comprising the nucleic acids, and recombinant techniques for the production of the antibodies, which may comprise culturing the host cell so that the nucleic acid is expressed and, optionally, recovering the antibody from the host cell culture or culture medium.

A variety of vectors are known in the art. Vector components can include one or more of the following: a signal sequence (that, for example, can direct secretion of the antibody), an origin of replication, one or more selective marker genes (that, for example, can confer antibiotic or other drug resistance, complement auxotrophic deficiencies, or supply critical nutrients not available in the media), an enhancer element, a promoter, and a transcription termination sequence, all of which are well known in the art.

Suitable host cells include prokaryote, yeast, or higher eukaryote cells. Suitable prokaryotes include eubacteria, such as Gram-negative or Gram-positive organisms, for example, Enterohacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas, and Streptomyces. In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available, such as Pichia, e.g. P. pastoris, Schizosaccharomyces pombe; Kluyveromyces, Yarrowia; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-I variant of Autographa californica NPV and the Bm-5 strain of Bombyx mori NPV.

However, interest has been greatest in vertebrate cells, and propagation of vertebrate cells in culture (tissue culture) has become routine. Examples of useful mammalian host cell-lines are Chinese hamster ovary cells, including CHOKI cells (ATCC CCL61) and Chinese hamster ovary cells/−DHFR (DXB-11, DG-44; Urlaub et al, Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Virol. 36: 59 (1977)]; baby hamster kidney cells (BHK, ATCC CCL 10); mouse Sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI38, ATCC CCL 75); human hepatoma cells (Hep G2, HB 8065); mouse mammary tumor (MMT 060562, ATCC CCL51); TR1 cells (Mather et al., Annals N.Y. Acad. Sci. 383: 44-68 (1982)); MRC 5 cells and FS4 cells.

The host cells can be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. Pat. Re. No. 30,985 can be used as culture media for the host cells. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements also can be included at appropriate concentrations that would be known to those skilled in the art. The culture conditions, such as temperature, pH, and the like, are those previously used with the host cell selected for expression, and will be apparent to the artisan.

The antibody composition can be purified using, for example, hydroxylapatite chromatography, cation or anion exchange chromatography, or preferably affinity chromatography, using the antigen of interest or protein A or protein G as an affinity ligand. Protein A can be used to purify antibodies that are based on human γ1, γ2, or γ4 heavy chains (Lindmark et al., J. Immunol. Meth. 62: 1-13 (1983)). Protein G is recommended for all mouse isotypes and for human γ 3 (Guss et al., 20 EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, 25 NJ.) is useful for purification. Other techniques for protein purification such as ethanol precipitation, Reverse Phase HPLC, chromatofocusing, SDS-PAGE, and ammonium sulfate precipitation are also possible depending on the specific binding agent or antibody to be recovered.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3-5, and more usually, at least 5 or 8-10 amino acids in a unique spatial conformation.

“Specifically binds” to or shows “specific binding” towards an epitope means that the antibody reacts or associates more frequently, and/or more rapidly, and/or greater duration, and/or with greater affinity with the epitope than with alternative substances.

In some embodiments, anti-HGF antibodies can be produced according to the methods described above. Alternatively, anti-HGF antibodies are commercially available, for example, Catalog No.: sc-57193, sc-71244, sc-166724, sc-1358, sc-7949, sc-1357, sc-1356, sc-13087, sc-34462, sc-34461, sc-53301, and sc-53478, from Santa Cruz Biotechnology, Santa Cruz, Calif., USA, and anti-HGF antibody L2G7 (Takeda-Galaxy Biotech). In some embodiments, anti-HGF antibodies, in particular antibodies to human HGF are described in U.S. Patent Application Publication No. 2011/0229462, Ser. No. 13/051481 filed on Mar. 18, 2011, and U.S. Application Publication No. 2005/0118643, Ser. No. 10/893,576 filed on Jul. 16, 2004, the disclosures of which are incorporated herein in their entireties.

(ii) Nucleic Acids

In some embodiments, HGF-binding reagents can include oligonucleotide probes that are operable to identify a Met-related cancer cell that is synthesizing HGF in an autocrine-signalling loop. The oligonucleotide probes can be used to identify Met-related cancer cells that contain DNA or RNA that encode HGF. For example, the oligonucleotide probes can include a collection of probes capable of detecting the level of expression of the HGF gene from different species, preferably human, as described herein.

One embodiment of the invention is a kit for determining the responsiveness of a Met-expressing tumor to Met inhibition. The kit includes one or more oligonucleotide probes capable of detecting the level of expression of HGF. In particular embodiments, the kits provided by the invention comprise two oligonucleotide probes having a nucleotide sequence of SEQ ID NOs: 1-2. In one embodiment, the HGF-binding agent comprises nucleic acid probes (e.g., oligonucleotide probes, polynucleotide probes) that specifically hybridize to an RNA transcript (e.g., mRNA, hnRNA) of a HGF gene as described herein. Such probes are capable of binding (i.e., hybridizing) to a target nucleic acid of complementary sequence through one or more types of chemical bonds, usually through complementary base pairing via hydrogen bond formation. As used herein, a nucleic acid probe can include natural (i.e., A, G, U, C or T) or modified bases (7-deazaguanosine, inosine, etc.). In addition, the bases in the nucleic acid probes can be joined by a linkage other than a phosphodiester bond, so long as the linkage does not interfere with hybridization. Thus, probes can be peptide nucleic acids in which the constituent bases are joined by peptide bonds rather than phosphodiester linkages.

Guidance for performing hybridization reactions can be found in Current Protocols in Molecular Biology, John Wiley & Sons, N.Y. (1989), 6.3.1-6.3.6, the relevant teachings of which are incorporated herein by reference in their entirety. Suitable hybridization conditions resulting in specific hybridization vary depending on the length of the region of homology, the GC content of the region, and the melting temperature of the hybrid. Thus, hybridization conditions can vary in salt content, acidity, and temperature of the hybridization solution and the washes. Complementary hybridization between a probe nucleic acid and a target nucleic acid involving minor mismatches can be accommodated by reducing the stringency of the hybridization media to achieve the desired detection of the target nucleic acid. In a particular embodiment, the nucleic acid probes in the kits of the invention are capable of hybridizing to RNA (e.g., mRNA) transcripts under conditions of high stringency.

In another embodiment, the HGF-binding agent comprises a pair of oligonucleotide primers that are capable of specifically hybridizing to an RNA transcript of a HGF gene as described herein, or a corresponding cDNA. Such primers can be used in any standard nucleic acid amplification procedure (e.g., polymerase chain reaction (PCR), for example, RT-PCR, quantitative real time PCR) to determine the level of the HGF RNA transcript in the biological sample. As used herein, the term “primer” refers to an oligonucleotide, which is complementary to the template polynucleotide sequence and is capable of acting as a point for the initiation of synthesis of a primer extension product. In one embodiment, the primer is complementary to the sense strand of a polynucleotide sequence and acts as a point of initiation for synthesis of a forward extension product. In another embodiment, the primer is complementary to the antisense strand of a polynucleotide sequence and acts as a point of initiation for synthesis of a reverse extension product. The primer can occur naturally, as in a purified restriction digest, or be produced synthetically. The appropriate length of a primer depends on the intended use of the primer, but typically ranges from about 5 to about 200; from about 5 to about 100; from about 5 to about 75; from about 5 to about 50; from about 10 to about 35; from about 18 to about 22 nucleotides. A primer need not reflect the exact sequence of the template but must be sufficiently complementary to hybridize with a template for primer elongation to occur, i.e., the primer is sufficiently complementary to the template polynucleotide sequence such that the primer will anneal to the template under conditions that permit primer extension.

In some embodiments, nucleic acids operable to bind to a HGF-gene or portions thereof under stringent conditions of hybridization include the oligonucleotides of SEQ ID NO:s 1-2. In an illustrative method of using the oligonucleotide HGF-binding reagents of the present invention, PCR using oligonucleotide HGF-binding reagent to detect HGF-autocrine Met-related cancer in a biological sample can be performed as follows: 5 μL of cDNA (RNA reverse transcribed into cDNA, the RNA extracted from a Met-related cancer biological sample from a subject diagnosed with or suspected of having a Met-related cancer) can be added to a reaction mixture containing 0.5 μM of each primer, 50 mM KCl, 10 mM TRIS-HCl (pH 8.3), 1.5 mM MgCl₂, 0.2 μM dNTPs and 1 U of Taq-polymerase (Perkin Elmer) in a total volume of 25 μL. The PCR can be run for 35 (for human HGF) cycles on a PCR thermocycler, with each cycle comprising 40 sec at 94° C., 40 sec at 62° C. (for human HGF), followed by 1 mM of primer extension at 72° C. The expected PCR products of approximate size of 749 bp (HGF) can be visualized by ethidium bromide staining on a 2% agarose gel.

(iii) HGF-Binding Proteins

In some embodiments, the HGF-binding reagent can include peptides and proteins that naturally bind to HGF. In some embodiments, illustrative HGF binding proteins can include cMet polypeptides that bind to HGF, for example, the human Met IPT domains 3 and 4. Polypeptides ranging in size from approximately 90 amino acids within each of these IPT domain 3 or 4 can be labeled with a fluorophore, radioisotope or other peptide tag, for example, 6-8×His, FLAG, GST, luciferase etc and used as a HGF-binding reagent. In one exemplary embodiment, the HGF-binding protein has an amino acid sequence comprising PIVYEIHPT KSFISGGSTI TGVGKNLNSV SVPRMVINVH EAGRNFTVAC QHRSNSEIIC CTTPSLQQLN LQLPLKTKAF FMLDGILSKY FDLIYV (SEQ ID NO: 13) or fragments thereof. (38)

D. Detectable Tags

In various embodiments, detectable tags can be attached, affixed, coupled, conjugated, labeled or otherwise connected with HGF-binding agents. Detectable tags can simplify detecting HGF level of expression when using a HGF-binding agent labeled or conjugated with a detectable tag. In some embodiments, HGF-binding agents can be attached, affixed, coupled, conjugated, labeled or otherwise connected with a fluorophore, a radioligand, a chemilluminescence molecule, a conjugated enzyme, a peptide conjugate molecule, and combinations thereof. Methods for affixing such detectable tags are commonly known in the art.

The probes in the kits of the invention can be conjugated to one or more labels (e.g., detectable labels). Numerous suitable detectable labels for probes are known in the art and include any of the labels described herein. Suitable detectable labels for use in the methods of the present invention include, but are not limited to, chromophores, fluorophores, haptens, radionuclides (e.g., ³H, ¹²⁵I, ¹³¹I, ³²P, ³³P, ³⁵S, ¹⁴C, ⁵¹Cr, ³⁶Cl, ⁵⁷Co, ⁵⁸Co, ⁵⁹Fe and ⁷⁵Se), fluorescence quenchers, enzymes, enzyme substrates, affinity tags (e.g., biotin, avidin, streptavidin, etc.), mass tags, electrophoretic tags and epitope tags that are recognized by an antibody (e.g., digoxigenin (DIG), hemagglutinin (HA), myc, GST, Hexa-HIS, FLAG). In certain embodiments, the detectable label is present on the 5 carbon position of a pyrimidine base or on the 3 carbon deaza position of a purine base of a nucleic acid probe.

In a particular embodiment, the detectable label that is conjugated to the HGF-binding reagent, for example, a nucleic acid or antibody or fragment thereof, is a fluorophore. Suitable fluorophores can be provided as fluorescent dyes, including, but not limited to fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) and N,N,N′,N′-tetramethyl-6-carboxyrhodamine (TAMRA), Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), CAL dyes, Carboxyrhodamine 6G, carboxy-X-rhodamine (ROX), Cascade Blue, Cascade Yellow, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, Carboxy-fluorescein (FAM), Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Oyster dyes, Pacific Blue, PyMPO, Pyrene, Rhodamine 6G, Rhodamine Green, Rhodamine Red, Rhodol Green, 2′,4′,5′,7′-Tetra-bromosulfone-fluorescein, Tetramethyl-rhodamine (TMR), Texas Red, and Texas Red-X. Nucleic acids can also be labeled using fluorescence emitting metals such as ¹⁵²Eu, or others of the lanthanide series. These metals can be attached to the antibody molecule using such metal chelating groups as diethylenetriaminepentaacetic acid (DTPA), tetraaza-cyclododecane-tetraacetic acid (DOTA) or ethylenediaminetetraacetic acid (EDTA).

In addition to the various detectable moieties mentioned above, the HGF-binding reagents in the methods and kits of the invention can also be conjugated to other types of labels, such as spectrally resolvable quantum dots, metal nanoparticles or nanoclusters, etc., which can be directly attached to a nucleic acid probe. As mentioned above, detectable moieties need not themselves be directly detectable. For example, they can act on a substrate which is detected, or they can require modification to become detectable.

### II. Method of Treating Glioblastoma Multiforme

The present inventors have investigated a set of GBM cell lines and found that all HGF-autocrine GBM cells displayed c-Met phosphorylation and served to predict in vivo GBM responsiveness to Met inhibition. HGF-paracrine stimulation can promote GBM tumor growth but does not significantly influence Met drug responsiveness. GBM models with amplified Met and EGFR showed responsiveness to EGFR inhibition but not to Met inhibition in vivo. HGF-autocrine tumors have an activated Met signaling pathway that may predict the sensitivity of Met-related cancer subjects to Met inhibitors.

Other embodiments of the present invention include a method of treating glioblastomamultiforme (GBM) in a subject in need thereof, the method comprising, administering a therapeutically effective dose of a MET inhibitor in combination with a therapeutically effective dose of a epithelial growth factor receptor (EGFR) inhibitor. In one embodiment, a method of treating glioblastomamultiforme (GBM) in a subject in need thereof, the method includes administering a therapeutically effective dose of SGX523 and a therapeutically effective dose of erlotinib. As used herein, a Met inhibitor is an agent that inhibits the activity of the receptor tyrosine kinase c-Met. As used herein, an EGFR inhibitor is an agent that decreases the activity of the EGFR tyrosine kinase. In certain embodiments, an EGFR inhibitor is a specific binding agent, for example, a small molecule. In certain embodiments, an EGFR inhibitor is an antibody. As used throughout the entire application a “subject” for the purposes of the methods and treatments as described herein includes human subjects and animal subjects, for example, animal subjects can include mammals.

Met Inhibitors:

In some embodiments, Met inhibitors can include SGX523 & SGX126 (SGX Pharmaceuticals), AMG102 (Amgen), ARQ197 (ArQule), JNJ-38877605 (Johnson and Johnson), PF-04217903, PHA665752 & PF2341066 (Pfizer), MP470 (SuperGen), MGCD265 (Methylgene), SU11274, SU 11271 & SU11606 (Sugen), Kirin, Geldanamycins, MGCD265 (MethylGene), HPK-56 (Supergen), MetMAb (Genentech, Inc.), ANG-797 (Angion Biomedica), CGEN-241 (Compugen), Metro-F-1 (Dompe), ABT-869 (Abbott Laboratories) and K252a. In one embodiment, the Met inhibitor is SGX523.

Epithelial Growth Factor Receptor Inhibitors:

In some embodiments, EGFR inhibitors can include gefitinib, erlotinib, PKI-166, EKB-569, GW2016, CI-1033 and an anti-erbB antibody such as trastuzumab and cetuximab. In one embodiment, the EGFR inhibitor is erlotinib.

Compositions and Dosages

In some embodiments, a pharmaceutical composition comprises a therapeutically effective amount of a Met inhibitor and a therapeutically effective amount of an EGFR inhibitor and together with at least one pharmaceutically acceptable diluent, carrier, solubilizer, emulsifier, preservative and/or adjuvant. In one embodiment, the Met inhibitor is SGX523 and the EGFR inhibitor is erlotinib. In some embodiments, the optimal pharmaceutical composition will be determined by one skilled in the art depending upon, for example, the intended route of administration, delivery format and desired dosage. See, for example, Remington's Pharmaceutical Sciences, 18^{th} Edition, A. R. Gennaro, ed., Mack Publishing Company (1990). In certain embodiments, such compositions may influence the physical state, stability, rate of in vivo release and rate of in vivo clearance of the inhibitors of the invention.

In some embodiments, a pharmaceutical composition is in the form of a dosage unit comprising an amount of a Met inhibitor and an amount of an EGFR inhibitor. Examples of such dosage units are tablets and capsules. In some embodiments, a pharmaceutical composition comprises an amount of a Met inhibitor and an amount of an EGFR inhibitor. In some embodiments, a pharmaceutical composition comprising an amount of a Met inhibitor and an amount of an EGFR inhibitor comprises the same amounts of a Met inhibitor and an EGFR inhibitor. In certain embodiments, a pharmaceutical composition comprising an amount of a Met inhibitor and an amount of an EGFR inhibitor comprises different amounts of a Met inhibitor and an EGFR inhibitor.

In some embodiments, a pharmaceutical composition comprises an amount of a Met inhibitor from about 1 to 2000 mg. and an amount of an EGFR inhibitor from about 1 to 2000 mg. In some embodiments, a pharmaceutical composition comprises an amount of a Met inhibitor from about 1 to 500 mg and an amount of an EGFR inhibitor from about 1 to 500 mg. In some embodiments, a pharmaceutical composition comprises an amount of a Met inhibitor from about 10 mg to 150 mg and an amount of an EGFR inhibitor from about 10 mg to 150 mg. In some embodiments, a pharmaceutical composition comprises an amount of a Met inhibitor from about 25 to 125 mg and an amount of an EGFR inhibitor from about 25 to 125 mg. In certain embodiments, a pharmaceutical composition comprises an amount of a Met inhibitor selected from about 25 mg, about 50 mg, about 75 mg, about 100 mg, about 150 mg, about 250 mg, about 350 mg, and about 500 mg. together with an amount of an EGFR inhibitor selected from about 25 mg, about 50 mg, about 75 mg, about 100 mg, about 150 mg, about 250 mg, about 350 mg, and about 500 mg.

Administration

In some embodiments, a pharmaceutical composition may be administered by any suitable route. In some embodiments, a pharmaceutical composition may be administered in the form of a pharmaceutical composition adapted to a desired route. In some embodiments, a pharmaceutical composition may be administered orally, mucosally, topically, rectally, pulmonarily such as by inhalation spray, or parenterally, including intravascularly, intravenously, intraperitoneally, subcutaneously, intramuscularly, intrasternally, and using infusion techniques.

A “therapeutically effective amount” is an amount of a Met inhibitor or an EGFR inhibitor that, when administered to a patient, ameliorates a symptom of the Met-related cancer. The amount of a compound or compounds (i.e., a Met inhibitor and an EGFR inhibitor) of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

“Treating” or “treatment” of a Met-related cancer, as used herein, includes (i) preventing the Met-related cancer from occurring in a human, i.e. causing the clinical symptoms of the cancer not to develop in an animal that may be exposed to or predisposed to the cancer but does not yet experience or display symptoms of the disease; (ii) inhibiting the Met-related cancer, i.e., arresting its development; and (iii) relieving the Met-related cancer, i.e., causing regression of the disease. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

### III. Kits

A kit for determining the responsiveness of a Met-related cancer to MET inhibition, is provided. In some embodiments, the kit includes: a container for collecting a biological sample from a subject and a HGF-binding reagent for detecting an HGF-autocrine cancer in the biological sample. In some embodiments, the HGF-autocrine cancer is HGF-autocrine glioblastoma multiforme.

In some embodiments of the present invention kits for identifying an HGF-autocrine Met-related tumor or tumor cells are provided. Kits of the invention include a collection container for collecting a subject's biological sample. Preferably, the container is sterile and its construction does not interfere in the assay to detect a HGF-autocrine cancer. The kit also includes one or more HGF-binding reagents. The HGF-binding reagents employed in the kits of the invention include, but are not limited to, nucleic acid probes and antibodies. Accordingly, in one embodiment, the kit comprises nucleic acid probes (e.g., oligonucleotide probes, polynucleotide probes) that specifically hybridize to an HGF RNA transcript (e.g., mRNA, hnRNA or cDNA thereof) of a HGF gene as described herein. Such probes are capable of binding (i.e., hybridizing) to a target nucleic acid of complementary sequence through one or more types of chemical bonds, usually through complementary base pairing via hydrogen bond formation. As used herein, a nucleic acid probe can include natural (i.e., A, G, U, C or T) or modified bases (7-deazaguanosine, inosine, etc.). In addition, the bases in the nucleic acid probes can be joined by a linkage other than a phosphodiester bond, so long as the linkage does not interfere with hybridization. Thus, probes can be peptide nucleic acids in which the constituent bases are joined by peptide bonds rather than phosphodiester linkages.

Guidance for performing hybridization reactions can be found in Current Protocols in Molecular Biology, John Wiley & Sons, N.Y. (1989), 6.3.1-6.3.6, the relevant teachings of which are incorporated herein by reference in their entirety. Suitable hybridization conditions resulting in specific hybridization vary depending on the length of the region of homology, the GC content of the region, and the melting temperature (“Tm”) of the hybrid. Thus, hybridization conditions can vary in salt content, acidity, and temperature of the hybridization solution and the washes. Complementary hybridization between a probe nucleic acid and a target nucleic acid involving minor mismatches can be accommodated by reducing the stringency of the hybridization media to achieve the desired detection of the target nucleic acid. In a particular embodiment, the nucleic acid probes in the kits of the invention are capable of hybridizing to RNA (e.g., mRNA) transcripts under conditions of high stringency.

In another embodiment, the kits include pairs of oligonucleotide primers that are capable of specifically hybridizing to an RNA transcript of a gene, or a corresponding cDNA. Such primers can be used in any standard nucleic acid amplification procedure (e.g., polymerase chain reaction (PCR), for example, RT-PCR, quantitative real time PCR) to determine the level of the RNA transcript in the sample. As used herein, the term “primer” refers to an oligonucleotide, which is complementary to the template polynucleotide sequence and is capable of acting as a point for the initiation of synthesis of a primer extension product. In one embodiment, the primer is complementary to the sense strand of a polynucleotide sequence and acts as a point of initiation for synthesis of a forward extension product. In another embodiment, the primer is complementary to the antisense strand of a polynucleotide sequence and acts as a point of initiation for synthesis of a reverse extension product. The primer can occur naturally, as in a purified restriction digest, or be produced synthetically. The appropriate length of a primer depends on the intended use of the primer, but typically ranges from about 5 to about 200; from about 5 to about 100; from about 5 to about 75; from about 5 to about 50; from about 10 to about 35; from about 18 to about 22 nucleotides. A primer need not reflect the exact sequence of the template but must be sufficiently complementary to hybridize with a template for primer elongation to occur, i.e., the primer is sufficiently complementary to the template polynucleotide sequence such that the primer will anneal to the template under conditions that permit primer extension.

In another embodiment, the kits of the invention include antibodies that specifically bind to HGF. Such antibody HGF-binding reagents, as described above, can be polyclonal, monoclonal, human, chimeric, humanized, primatized, veneered, or single chain antibodies, as well as fragments of antibodies (e.g., Fv, Fc, Fd, Fab, Fab′, F(ab′), scFv, scFab, dAb), among others. (See e.g., Harlow et al., Antibodies A Laboratory Manual, Cold Spring Harbor Laboratory, 1988).

The HGF-binding reagents as discussed above can be used in the methods of the present invention to detect whether the Met-related cancer collected in the container from a subject is HGF-autocrine. In some embodiments, the HGF-binding reagent can include HGF specific primers (e.g., one or more) or probes capable of detecting the expression level of HGF in a biological sample described herein.

In one embodiment, a biological sample can include a fresh or frozen tumor biopsy from a subject diagnosed with or suspected of having a met-related cancer. The HGF specific primers can be used to perform RT-PCR with isolated nucleic acids from the biological sample. If the HGF identified with the HGF specific primers is expressed from the met-related cancer in an autocrine fashion, the met-related cancer is sensitive to Met inhibition. In some embodiments, the same biopsied sample can be confirmed to be HGF-autocrine by processing the biopsied tumor to make formalin fixed paraffin embedded tissue blocks. Once the biopsy sample is sectioned, immunohistochemical staining, using anti-HGF antibodies or HGF-binding fragments thereof, a medical practitioner can review the slides and confirm whether the HGF staining represents HGF-autocrine.

### EXAMPLES

### Example 1

### Materials and Methods

A. Cell culture.

DBM2, U251M2, and U87M2 are the invasive subclones generated from the human glioblastoma multiforme cell lines DBTRG-05MG, U251, and U87, as described in the inventors' previous study (20); the U118, SF295, SF268, and SF539 lines were from NCI-60. To select faster growing subpopulations from the SF295 line, cells (5×10⁵ in 100 μl PBS) were inoculated into SCID_{hgf} mice subcutaneously to form tumors. The fast growing tumor was selected for primary tissue culture as described previously (20). Briefly, a SF295 SQ tumor was harvested at necropsy, washed in PBS, minced and treated with 0.25% trypsin (Invitrogen) for 45 min. The cells (SF295SQ1) were collected by low-speed centrifugation and resuspended in complete DMEM containing 10% FBS. T98G was provided by Dr. Shinomiya Nariyoshi of the National Defense Medical College, Japan. These cell lines were grown in DMEM (Gibco™, Invitrogen Corporation) supplemented with 10% FBS (Hyclone), and 1% penicillin, and streptomycin (Invitrogen Corporation). X01-GB is a GBM stem cell line provided by Dr. Akio Soeda of the University of Gifu, Japan, and was grown with Neurobasal medium and supplemented with B27, EGF (20 ng/ml), bFGF (20 ng/ml) and 1% penicillin and streptomycin (Gibco, Invitrogen Corporation). SGX523 was provided by Lily Pharmaceuticsand. Erlotinib was provided by OSI Pharmaceuticals. For in vitro study, compounds were diluted in dimethyl sulfoxide (DMSO) at 0.01 M, separated into small aliquots (5 μl), and kept at −80° C. until use.

B. HGF-Induced uPA Activity.

Cells were grown overnight in DMEM/10% FBS. Drugs were dissolved in DMSO and serially diluted from stock concentrations into DMEM/10% FBS medium and added to the appropriate wells. Immediately after drug or reagent addition, HGF/SF (60 ng/ml) was added to all wells (with the exception of wells used as controls to calculate basal growth and uPA-plasmin activity levels). Twenty-four hours after drug and HGF/SF addition, plates were processed for the determination of plasmin activity as described previously (22). Wells were washed twice with DMEM (without phenol red; Life Technologies, Inc.), and 200 μl of reaction buffer [50% (v/v) 0.05 units/ml plasminogen in DMEM (without phenol red), 40% (v/v) 50 mM Tris buffer (pH 8.2), and 10% (v/v) 3 mM Chromozyme PL (Boehringer Mannheim) in 100 mM glycine solution] was added to each well. The plates were then incubated at 37° C., 5% CO, for 4 h, at which time the absorbance was read on an automated spectrophotometric plate reader at the single wavelength of 405 nm.

C. HGF-Induced Downstream Signaling Pathway.

Cells were seeded in 10-cm dishes and grown until 80% confluent. After serum starvation overnight, cells were treated with or without SGX523 for 4 h with (or without) human HGF/SF or EGF (100 ng/ml) for 20 minutes at 37° C. The cells were washed twice with ice-cold 1×PBS, and whole cell lysates were prepared using RIPA buffer. The protein concentrations were determined by DC protein assay. Equal amounts of total protein (30 μg) from cell lysates were loaded on a 4-20% SDS-PAGE gel (invitrogen), transferred to a polyvinylidene difluoride (PVDF) membrane (Invitrogen), and detected using an ECL Western Blotting Detection System (GE Healthcare). Antibodies used were human Met (clone 25H2,), phospho-Met (Y1234/1235), human EGFR (clone D38B1), phosphor-EGFR(Y1068), AKT, phospho-AKT (S473), p42/44 MAPK, phospho-p42/44 MAPK (T202/Y204) (all from Cell Signaling Technology); β-actin (clone AC-15, Abcam); and anti-HGFantibody (Clone 7-2, provided by Dr. Brian Cao at Van Andel Research Institute). Secondary antibodies used were goat anti-rabbit IgG-HRP and goat anti-mouse IgG-HRP (Santa Cruz Biotechnology).

D. GBM Patient-Derived Xenograft Tumor Model.

The V13 GBM xenograft tumor line was generated from the primary tumor of a GBM patient upon surgical removal. The fresh tumor specimen was split into two pieces, one to be divided and implanted into 5 nude mice to propagate tumor growth and another piece to be directly digested with 0.05% trypin into single cells and grown in Neuro-basal medium with 10% FBS supplemented with EGF (20 ng/ml), bFGF (20 ng/ml) and 1% penicillin and streptomycin at 37° C. The tumors growing from nude mice were analyzed using FISH to compare them with the original tumor and were further transplanted into the host nude mice to maintain tumor growth in vivo. All studies involving human subjects and human tissues were approved by IRB committee of Van Andel Research Institute.

E. RNA Preparation, Amplification, and Labeling for Microarray Analysis.

Total RNA was extracted from cells using Trizol (Invitrogen, Carlsbad, Calif.). RNA integrity was assessed using an Agilent 2100 Bioanalyser (Agilent Technologies, Waldbronn, Germany). Total RNA (3 μg) from the cells was amplified into anti-sense RNA (aRNA). Total RNA from peripheral blood mononuclear cells (PBMC) pooled from the 6 normal donors was extracted and amplified into aRNA to serve as the reference. Pooled reference and test aRNA were isolated and amplified under identical conditions and the same amplification/hybridization procedures to avoid possible interexperimental biases (Wang et al. 2005). Both reference and test aRNA were directly labeled using the ULS Fluorescent Labeling kit (Kreatech, Amsterdam, Netherlands) with Cy3 for reference and Cy5 for test samples. The two labeled aRNA probes were separated from unincorporated nucleotides by filtration, fragmented, mixed, and co-hybridized to custom-made 36 K oligoarrays at 42° C. for 24 h. The oligo-chips were printed at the Infectious Disease and Immunogenetics Section Department of Transfusion Medicine, Clinical Center, National Institutes of Health (Bethesda, Md.). After hybridization the arrays were then washed and scanned on a GenePix scanner Pro 4.0 (Axon, Sunnyvale, Calif.) with a variable photomultiplier tube to obtain optimized signal intensities with minimum (<1% spots) intensity saturation.

F. Data Processing and Statistical Analyses.

The raw data set was filtered according to a standard procedure to exclude spots below a minimum intensity that arbitrarily was set to an intensity parameter of 100 in both fluorescence channels. Spots with diameters<10 μm and flagged spots were also excluded from the analysis. The filtered data was then normalized using the median over the entire array and retrieved by the BRB-ArrayTools http://linus.nci.nih.gov/, which was developed at the National Cancer Institute (NCI), Biometric Research Branch, Division of Cancer Treatment and Diagnosis.

The samples were analyzed using paired Student's t-test. All analyses were tested for an univariate significance threshold set at a p-value<0.05. Gene clusters identified by the univariate t-test were challenged with two alternative additional tests, an univariate permutation test (PT) and a global multivariate PT. The multivariate PT was calibrated to restrict the false discovery rate to 10%. Genes identified by univariate t-test as differentially expressed (p-value<0.05) and a PT significance<0.05 were considered truly differentially expressed.

Hierarchical cluster analysis and TreeView software were used for visualization of the data. Gene annotation and functional pathway analysis was based on the Database for Annotation, Visualization and Integrated Discovery (DAVID) 2007 software and the Gene Cards website http://www.genecards.org/index.shtml.

The gene set expression comparison kit implemented in BRB-ArrayTools was used to perform a functional gene network analysis using the Ingenuity pathway analysis system. The Ingenuity Pathways Analysis (IPA) is a system that transforms large data sets into a group of relevant networks containing direct and indirect relationships between genes based on known interactions in the literature.

G. Fluorescence In Situ Hybridization (FISH).

FISH probes were prepared from the purified RP11-371D15 and RP11-34D10 at locus 4q25 spanning gene EGF, RP5-1091E12 and RP11-708P5 at locus 7p11.2 spanning gene EGFR, RP11-24F4 and RP11-24F4 at locus 7q21.11 spanning gene HGF, and RP11-163C9 and RP11-564A14 at locus 7q31.2 spanning gene MET (BACPAC Resource Center (BPRC), bacpac.chori.org). All DNA extracted from these BAC clones were directly labeled with SpectrumGreen or SpectrumOrange with the use of a nick translation kit and dUTP dyes from Abbott Molecular Inc. Metaphase slides were prepared from cells grown in culture, arrested in metaphase, and fixed in methanol:acetic acid (3:1) following standard cytogenetic harvest procedures. Sample slides were pretreated in 2× saline/sodium citrate (SSC) at 37° C. for 10 min, 0.005% pepsin/0.01 M HCl at 37° C. for 4 min, and 1×PBS for 5 min. The slides were then placed in 1% formaldehyde for 10 min at room temperature, washed with 1×PBS for 5 min, and dehydrated in an ethanol series (70%, 85%, and 95%) for 2 min each. Sample slides were denatured in 70% formamide/2×SSC at 74° C. for 3 min, washed in a cold ethanol series (70%, 85%, 95%) for 2 min each, and air-dried. FISH probes were denatured at 75° C. for 5 min and kept at 37° C. for 10-30 min. Eight microliters of probe was applied onto each slide and mounted with a glass coverslip. The slides hybridized overnight at 37° C., washed with 2×SSC at 73° C. for 2 min, and rinsed shortly in distilled water. Slides were air-dried, counterstained with VECTASHIELD mounting medium with 4′-6-diamidino-2-phenylindole (DAPI) (Vector Laboratories Inc., Burlingame, Calif.), and coverslips were applied. Image acquisition was performed with a COOL-1300 SpectraCube camera (Applied Spectral Imaging-ASI, Vista, Calif.) mounted on an Olympus BX51 microscope. Images were analyzed using FISHView EXPO v6.0 software (ASI), and at least 50-200 cells were scored for each sample.

H. RT-PCR.

Total RNA was extracted from cells using Trizol reagent (Invitrogen) according to the manufacturer's instructions. Total RNA (500 ng) was used for each reaction (25 μl) using the OneStep RT-PCR Kit (qiagen) according to the manufacturer's instructions. Primers used in RT-PCR reactions included:

human HGF
(SEQ ID NO: 1)
forward: 5′-CAGCGTTGGGATTCTCAGTAT-3′,
(SEQ ID NO: 2)
reverse: 5′-CCTATGTTTGTTCGTGTTGGA-3′;
human MET
(SEQ ID NO: 3)
forward: 5′-ACAGTGGCATGTCAACATCGCT-3′,
(SEQ ID NO: 4)
reverse: 5′-GCTCGGTAGTCTACAGATTC-3′;
β-actin
(SEQ ID NO: 5)
forward: 5′-GGCGGCAACACCATGTACCCT-3′,
(SEQ ID NO: 6)
reverse: 5′-AGGGGCCGGACTCGTCATACT-3′;
human EGFR
(SEQ ID NO: 7)
forward: 5′-CTTCTTGCAGCGATACACTGC-3′,
(SEQ ID NO: 8)
reverse, 5′-ATGCTCCAATAAATTCACTGC-3′;
human EGFRvIII
(SEQ ID NO: 9)
forward: 5′-ATGCGACCCTCCGGGACG-3′,
(SEQ ID NO: 10)
reverse: 5′-ATTCCGTTACACACTTTGCGGC-3′;

designed to flank the deletion of exons 2 to 7 (39). Reverse transcription was done at 50° C. for 30 min followed by enzyme inactivation and hot-start PCR at 95° C. for 15 min. Denaturation, annealing, and extension were done at 94° C., 57° C., and 72° C., respectively, for 1 min each for a total of 25 cycles. The reaction was completed with an extension period at 70° C. for 10 min. Five microlitres of the RT-PCR product was run on a 1.3% agarose gel.

I. In Vivo SGX523 and Erlotinib Therapeutic Efficacy Study.

All animal studies were approved by the IACUC of the Van Andel Research Institute. Each GBM cell line (5×10⁵ cells in 100 μl PBS) was inoculated into both SCID and SCIDhgf mice subcutaneously. For V13 tumor line, tumors were transplanted into SCIDhgf mice at the flank to initiate tumor growth. Tumor size was measured with a caliper twice a week. When average tumor size reached 100 mm³, mice were grouped (n=10) for treatment as indicated. Dosing with SGX523 and/or erlotinib was delivered once daily by oral gavage for 3 weeks. Vehicles used were 0.5% MC 400 with 0.05% Tween 80 (SGX523) and 0.5% (w/v) methyl cellulose (erlotinib). Tumor size was measured by a caliper twice a week. Body weight was measured once a week. All mice were sacrificed 24 h after last dose. To determine the effectiveness of treatment, the average tumor size of each group from the last measurement was analyzed with Student's t test (α=0.05).

J. ELISA Analysis.

At the end of the efficacy study, mouse serum was collected from each group and kept at −80° C. until use. For HGF expression level, 50 μl of serum was used for an ELISA test (R&D) according to the kit instructions. At least five samples were tested. Data represent mean±SD. Student's t test was used for statistical analysis.

K. In Silico Analysis of TCGA Datasets.

(i) Patients and Tumor Samples.

A large set of glioblastoma and normal brain samples was collected and processed through the TCGA Biospecimens Core Resource at the International Genomics Consortium (Phoenix, Ariz.). Gene expression profile was assayed using Affymetrix U133A, Affymetrix Exon 1.0 ST and custom Agilent 244K. DNA copy number analyses were performed using the Agilent 244K, Affymetrix SNP6.0, and Illumina 550K DNA copy number platforms. (TCGA, Nature, 2008).

In the present study, based on criteria elsewhere described (Roel G W Verhaak et al Cancer Cell 2010), 200 GBMs and 2 normal samples (derived from patient with epilepsy) were selected and analyzed using data derived from TCGA (TCGA, Nature 2008). Custom Agilent 244K and Agilent 244K copy number platforms were considered for expression and copy number analysis, respectively.

(ii) Gene Expression Analysis.

Transcriptional data were uploaded to the mAdb databank (http://nciarray.nci.nih.gov) and further analyzed using BRBArrayTools http://linus.nci.nih.gov/BRB-ArrayTools.html (Simon et al., 2007), Partek Genomics Suite (St Louis, Mo.), Stanford Cluster Program and TreeView software (40, 41). A self organizing heat map displaying HGF, EGFR and MET genes segregated the 202 GBM samples in 4 groups with specific HGF, MET and EGFR characterizations. Gene ratios were average corrected across experimental samples and displayed according to uncentered correlation algorithm.

(iii) Array Comparative Genomic Hybridization (CGH) Analysis

Copy number data for 183 of the 202 samples were examined for correlations with transcriptional profiles. Data described by Cancer Genome Atlas research Network (2008) were imported into Partek Genomic Suite and analyzed using the Copy Number Analysis workflow. The 4 different groups observed by transcriptional analysis were used in the analysis as a categorical parameter. In the analysis, significantly different regions were determined using the Segmentation Model algorithm of the Partek Genomic Suite set to detect copy number states. Segments are defined as regions that differ from neighboring regions by at least 2 signals to noise ratios in at least 10 markers. Amplifications were defined as segments with log2 intensity ratios greater than 0.15. Regions identified were annotated with gene symbols by importing the annotation file from the NCBI RefSeq genome browser (build Hg19).

### Example 2

### HGF Expression Correlates with MET Phosphorylation in HGF-Autocrine Glioblastoma

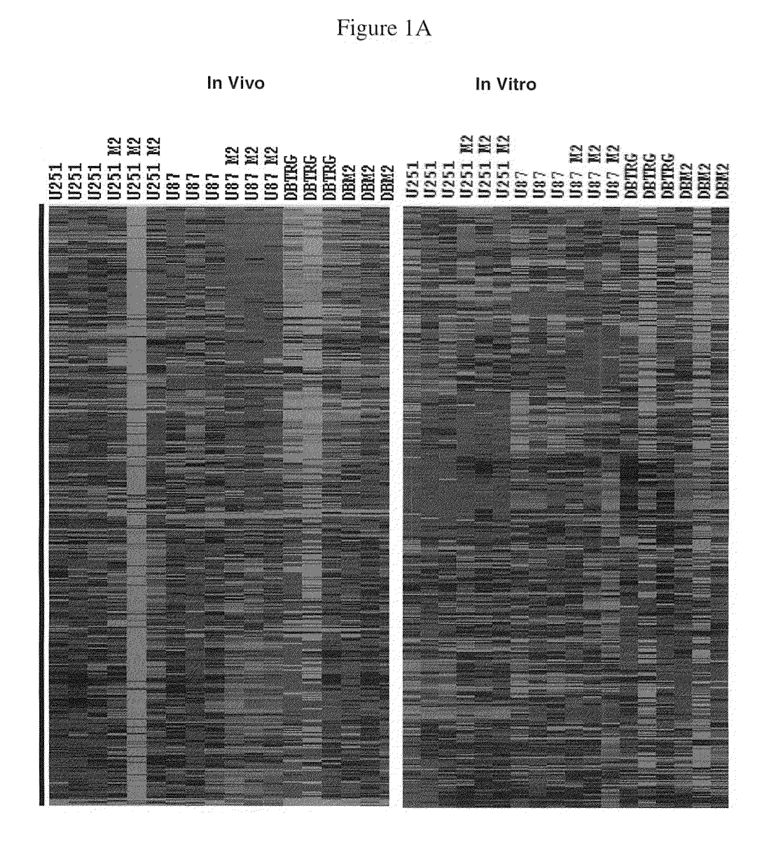

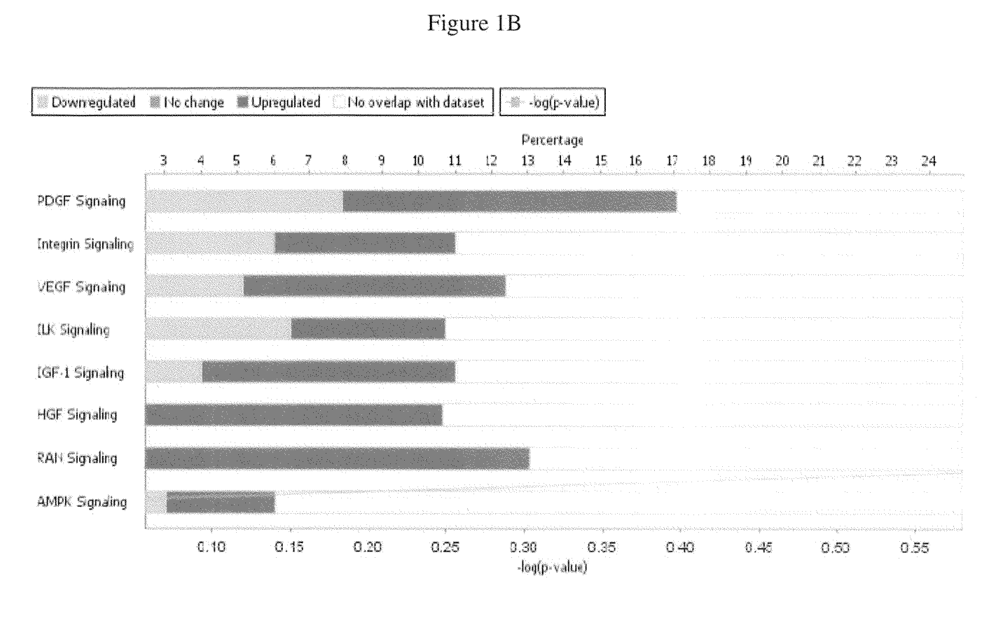

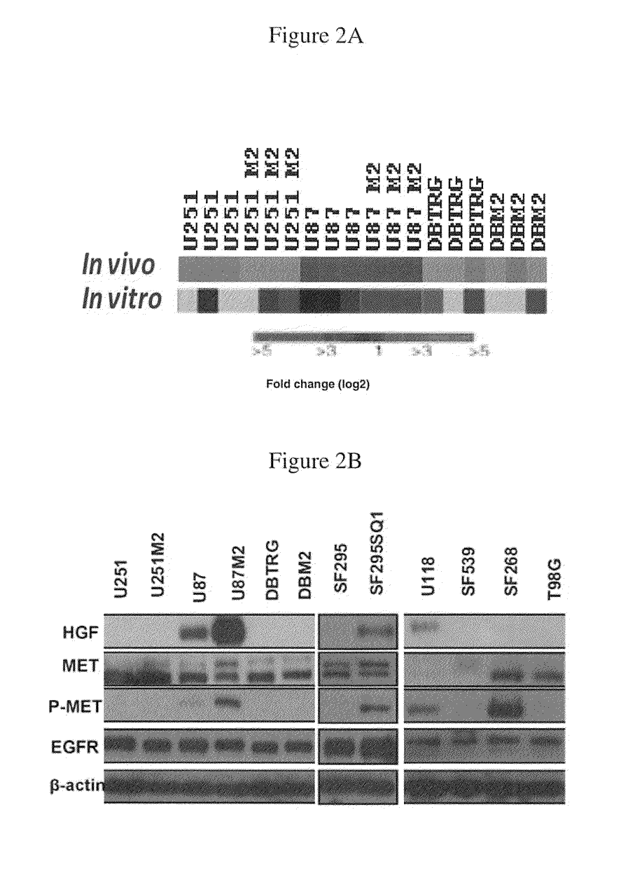

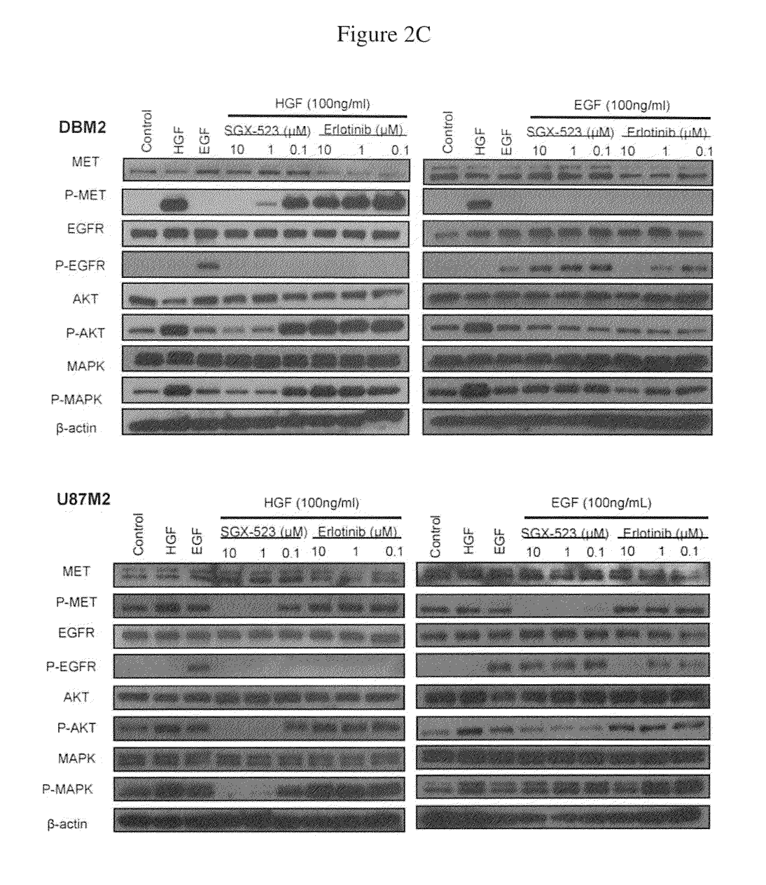

The inventors previously studied the GBM invasion by examining its metastatic potential. The inventors found that commonly used GBM cell lines (U251, U87, and DBTRG-05MG) have subpopulations with metastatic potential that can be selected via experimental metastasis assay. Compared with the parental cells, these metastatic sub-lines (U251M2, U87M2, DBM2), not only induced lung metastatic lesions, but also grew more aggressively with reduced survival times in orthotopic mouse models. At the molecular level, the M2 derivatives showed elevated IL-6, Il-8, GM-CSF, and BDNF, factors associated with either cancer metastasis or GBM malignancy (20). To identify further candidate targets playing a role in glioma invasion, the inventors used microarray technology to compare the GBM-M2 lines DBM2, U87M2, and U251M2 to their parental lines both in vitro and in an in vivo orthotopic GBM model. A paired analysis identified 1,008 genes differentially expressed in vitro (cutoff p-value<0.05 in a paired student t test, multivariate permutation test=0.06) between the three GBM-M2 lines and their respective parental lines (FIG. 1A). The same analysis identified 1,764 genes differentially expressed between in vivo-derived intracranial tumors and the parental lines (multivariate permutation test p-value=0.008; FIG. 2A). The inventors found that 206 genes were expressed in common both in vitro and in vivo analyses. It is well-known that HGF activates the Met signaling pathway and induces invasive tumor growth mainly through the MAPK and Akt pathways (1). Interestingly, while HGF transcription was most significantly elevated relative to the parental cell line, U87M2, both in vivo and in vitro. (FIG. 2A), the receptor Met level remained unchanged. Since intracranial tumors likely provide a better representation of GBM tumor biology, the inventors used in vivo microarray data to further analyze HGF signaling pathways potentially responsible for glioma invasiveness. The average expression values of the genes of the three cell lines reported in the FIG. 1A was applied with Ingenuity pathway analysis. Particularly with in vivo data, the inventors observed that HGF signaling counts as one of the top 8 canonical pathways associated with cancer signaling and cell growth pathways, and is the one with the most genes up-regulated (FIG. 1B,). Enhanced HGF transcription resulted in significant up-regulation of the RAS-MAPK and AKT pathways, the leading pathways involved in gliomagenesis (6, 11), without affecting MET transcriptional levels. These observations suggested that it could be the endogenous HGF expression, rather than MET expression, determines the MET signaling activity in this model.

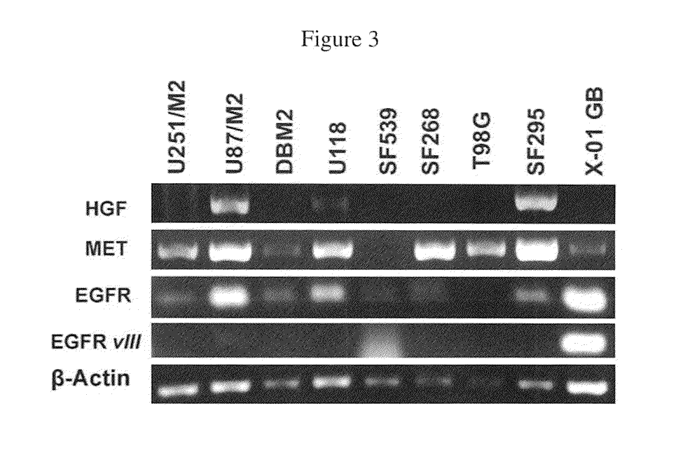

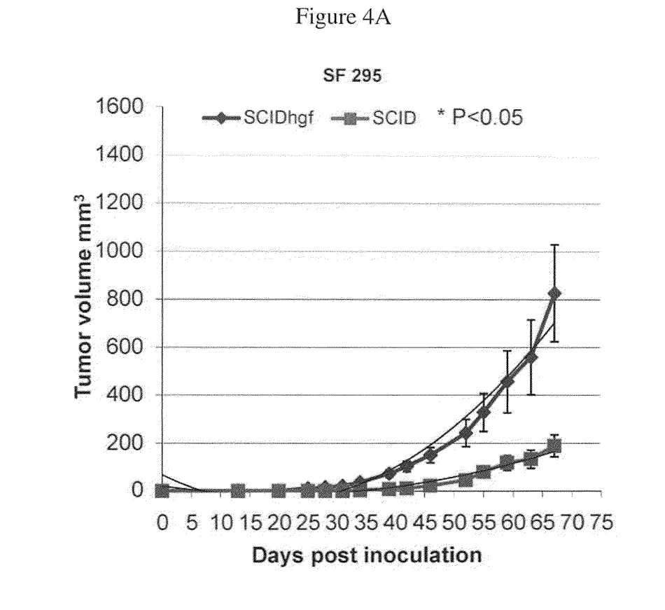

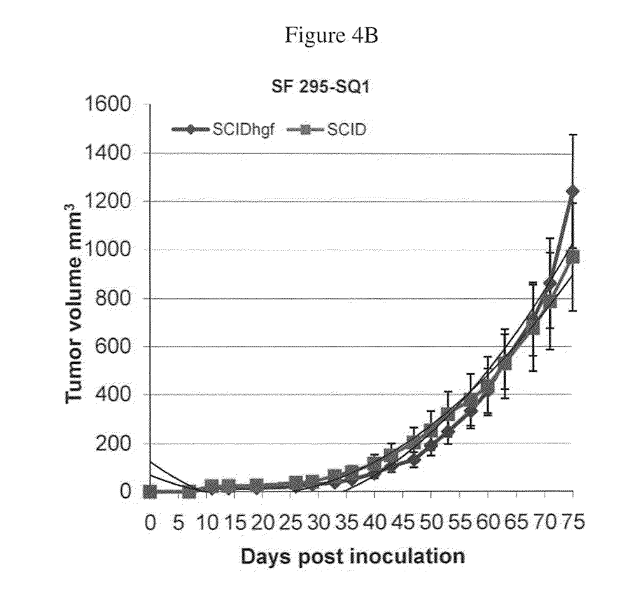

To validate the microarray results, the inventors compared the three pairs of GBM cell lines in terms of HGF and MET expression levels with Western blot. The up-regulation of U87M2 is consistent with the elevated HGF expression in the microarray results (FIG. 2B) More importantly, U87M2 cells also displayed higher levels of p-MET relative to the U87 parental cells, indicating that HGF may be the primary regulator of MET activity in autocrine tumors. The inventors also screened a set of GBM cell lines by western blot (FIG. 2B) and RT-PCR (FIG. 3) to compare the expression levels of HGF, MET, and p-MET. SF295SQ1 is a sub-line from SF295 characterized by enhanced tumor growth in SCID, mice (FIG. 4A), showed selection for elevated HGF with little or no change in MET expression, (FIG. 2B) indicative of becoming more autocrine for HGF. The inventors observed that U87M2, SF295SQ1, and U118 expressed HGF at levels comparable to the levels of p-MET (FIGS. 2B and 3); in contrast, MET expression showed no correlation with either HGF or p-MET. Interestingly, SF268 displayed p-MET activity with no HGF secretion, suggesting ligand-independent activation. These data suggest that in GBM with HGF-autocrine expression, it is the HGF level, rather than that of MET expression, that is associated with Met signaling activation. Based on these results, the inventors asked whether HGF-autocrine expression determines sensitivity to MET inhibition.

### Example 3

### HGF-Autocrine Activation Display High Sensitivity to Met Inhibitors

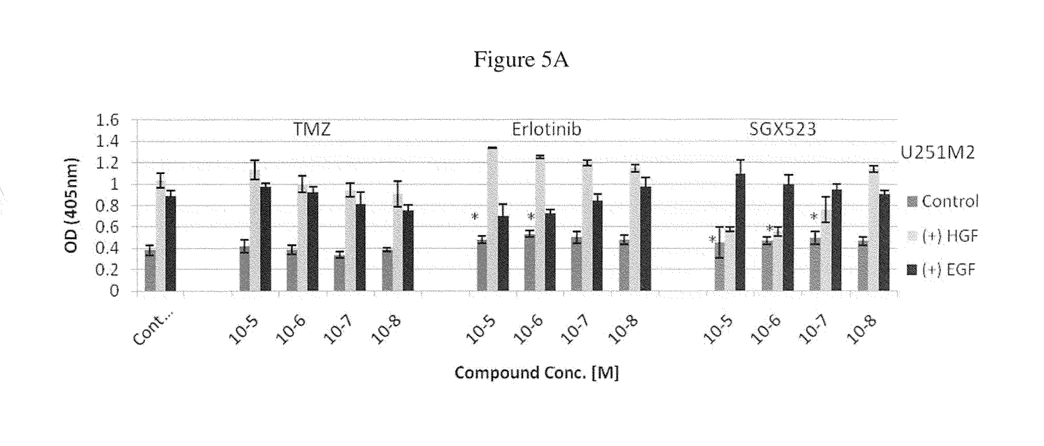

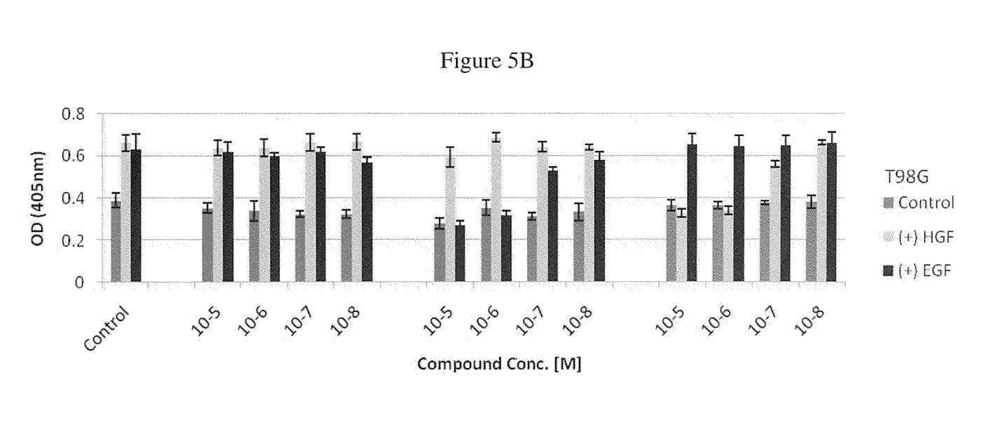

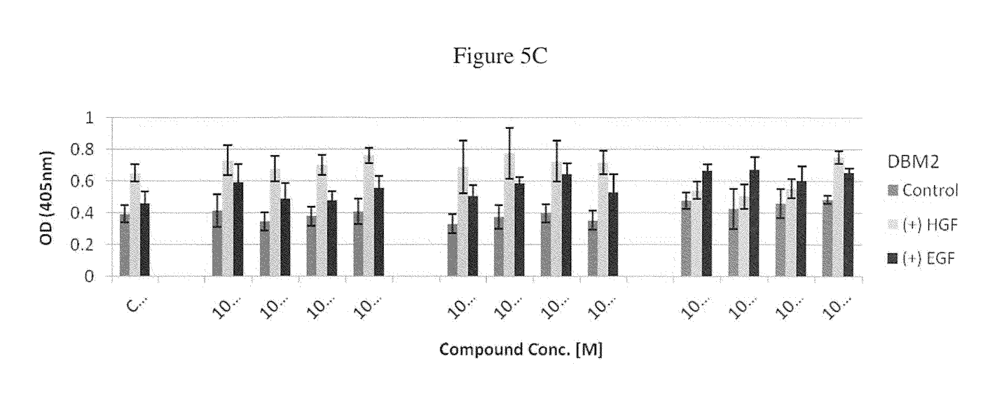

HGF can enhance GBM invasion by up-regulating urokinase (uPA) activity. Previously, a uPA assay was established as a surrogate assay to quantify HGF-induced invasion in vitro (21, 22). The inventors compared the response of the GBM cell lines to HGF stimulation and to the inhibitory effect of the MET inhibitor SGX523 in vitro. The inventors show that HGF up-regulated the uPA activity in U251M2, T98G, and DBM2 cells (FIGS. 5A-5D), which do not display autocrine HGF expression. SGX523 inhibited HGF-induced uPA activity at 0.1 μM after 24 h. EGF also up-regulated uPA activity in these cells, which was specifically blocked by the EGFR inhibitor erlotinib within 24 hrs. However, there was no evidence of cross inhibition between SGX523 and Erlotinib, indicating the two signaling pathways are distinct from each other, at least at early time point in vitro. By contrast, U87M2 cells did not respond to either HGF or EGF, but SGX523 did inhibit the basal level of uPA activity even when the cells were stimulated by EGF, indicating that U87M2 cells are highly dependent on endogenous HGF activation. In all cases, temozolimide (TMZ), a standard therapeutic chemo reagent used in treating GBM, did not show activity inhibiting uPA at 24 hrs, showing that SGX523 or erlotinib work independent of TMZ in the mechanism of action.

Western blot analysis showed results consistent with the uPA assay (FIG. 2C). With DBM2 cells, both HGF and EGF activate MAPK and AKT pathways through phosphorylation of their respective receptors. Again, there is no evidence of cross inhibition between the MET and EGFR pathways, since SGX523 did not inhibit EGF induced p-EGFR, nor did Erlotinib inhibit HGF induced p-MET. The inventors' data also supported the concept that U87M2 is highly dependent on the HGF-activated downstream signaling pathway (FIG. 2C). Compared with DBM2, which requires external HGF stimulation to initiate the downstream signaling pathways, U87M2 displays constitutive MET activation in the absence of HGF stimulation. At a 1-10 μM concentration, SGX523 can inhibit p-Met and downstream MAPK and AKT pathways regardless of additional HGF or EGF stimulation. These results indicate that HGF-autocrine level can be the dominant determinant of MET signaling pathway activity.

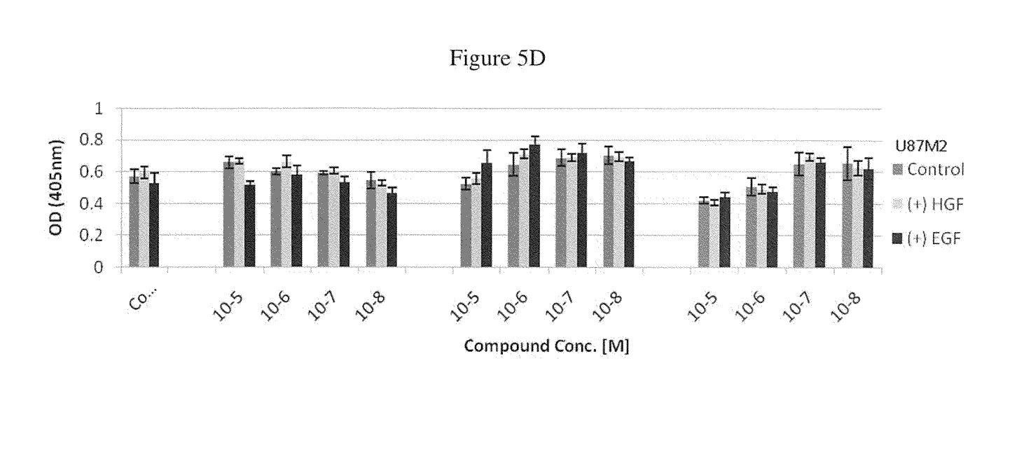

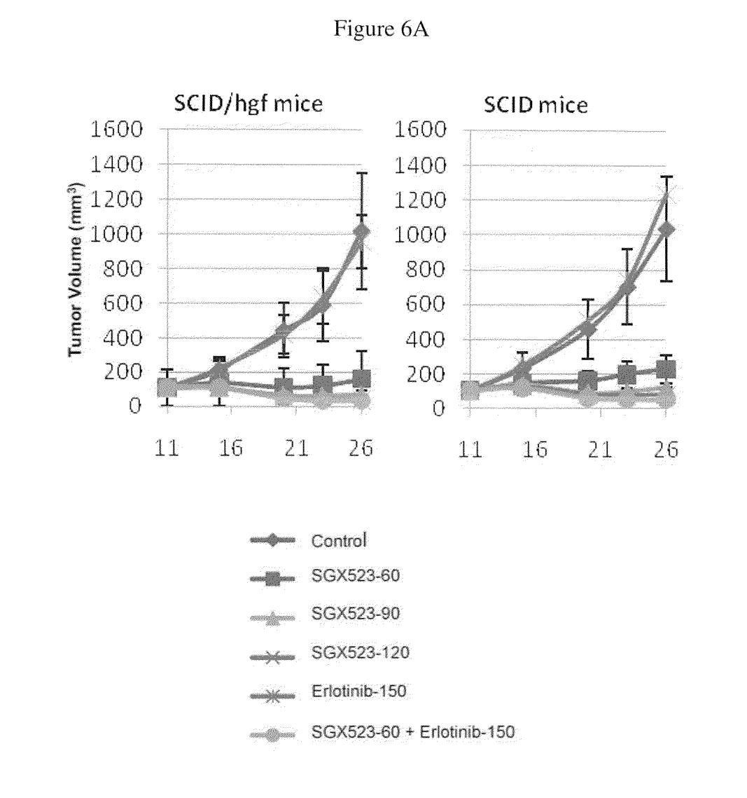

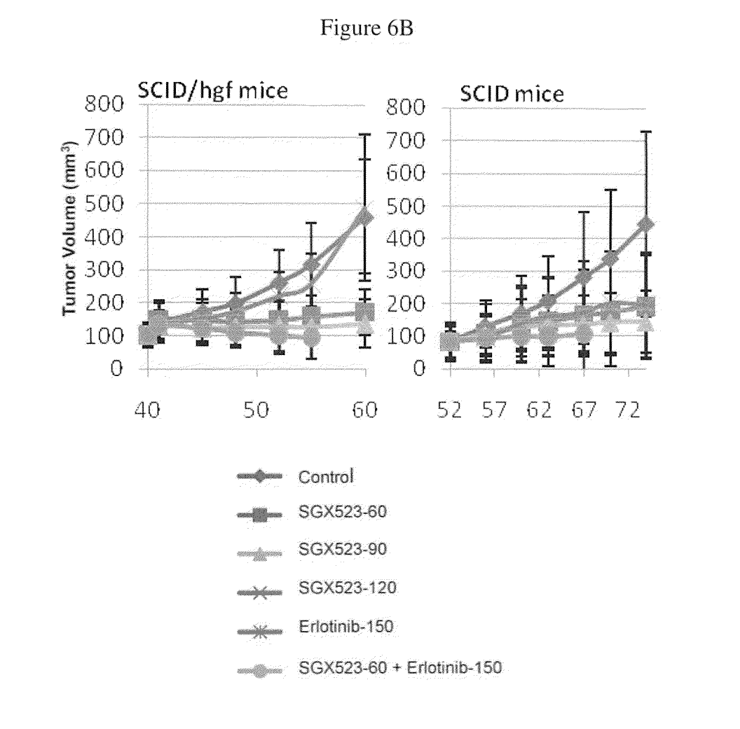

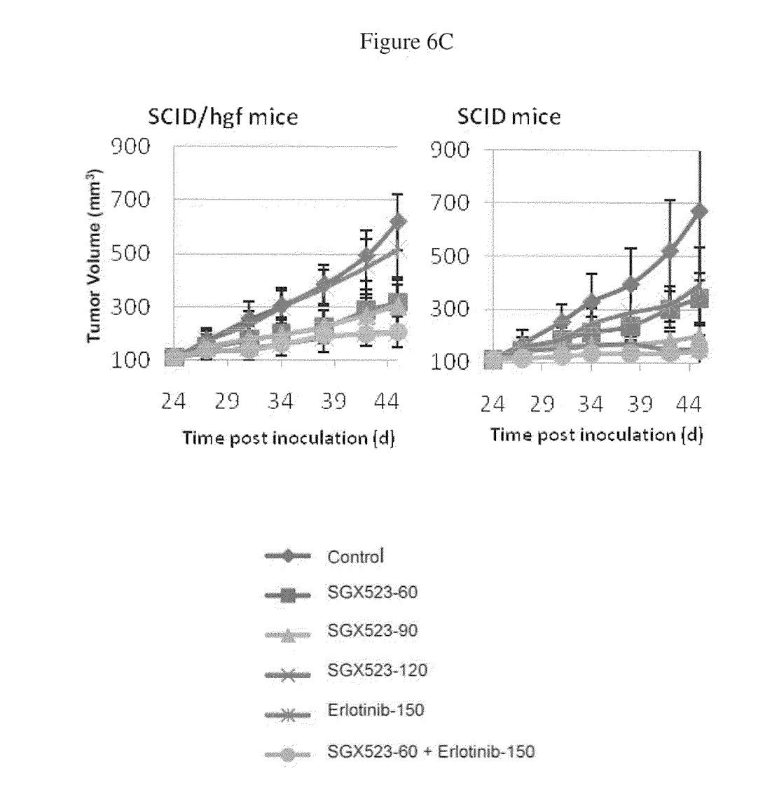

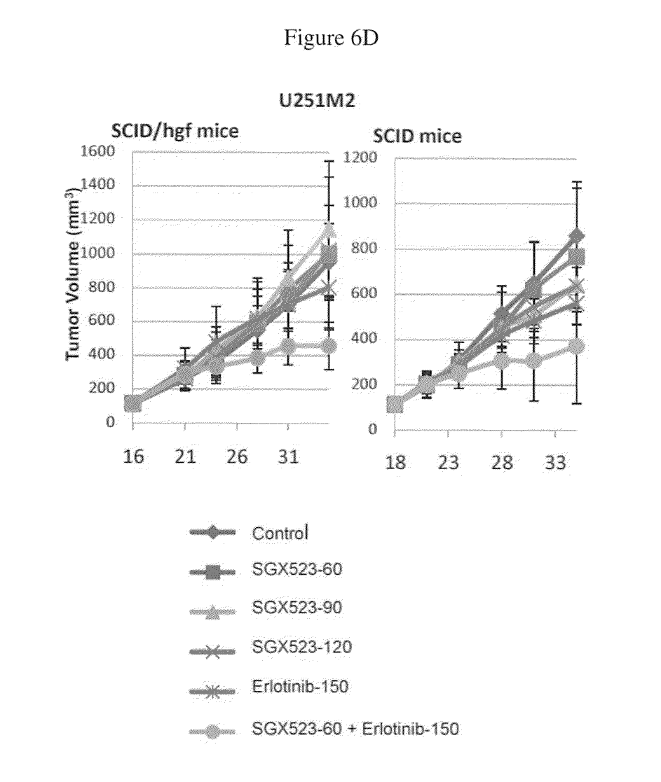

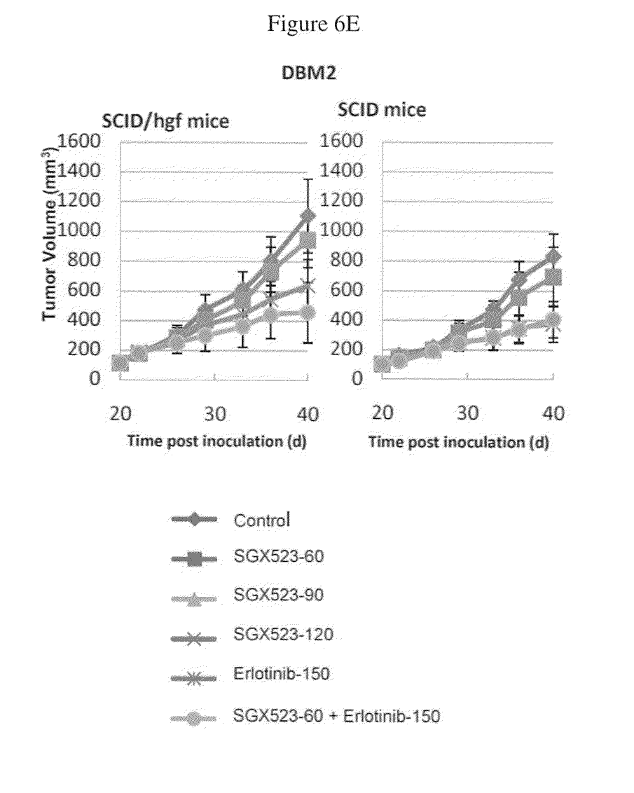

To test if the presence of an active HGF-autocrine loop predicts sensitivity to MET inhibitors in vivo, the inventors inoculated GBM cell lines (either with or without HGF-autocrine status) into mice subcutaneously and the inventors compared their sensitivity to SGX523. HGF-autocrine xenograft tumors from U87M2, U118, and SF295SQ1 (FIG. 6A-6C) were all sensitive to SGX523 in a dose-dependent manner (P<0.05 at all three doses). SGX523 caused dramatic tumor inhibition and regression within two weeks, showing that the HGF/Met autocrine activation plays a dominant role in tumor growth. These results indicate that HGF-autocrine status may be used as a predictive marker for targeting GBM with MET-inhibitors.

HGF-paracrine activation was evaluated by comparing xenograft tumor growth with and without expression of human HGF in SCIDhgf-Tg vs. SCID mice (23, 24). The HGF-autocrine U87M2 and SF295SQ1 cells displayed similar tumor growth potential in both types of mice (FIG. 6A-6C), while the GBM cells lacking HGF-autocrine activity showed partial growth advantage to paracrine HGF stimulation in SCIDhgf mice (unpaired t test, unequal variance DBM2:p<0.05, U251M2:p>0.05, FIG. 6D-6E), but showed no response to SGX523 (60 mg/kg). In the U251M2 model, increasing the dose to 120 mg/kg did not improve the responsive (FIG. 6D). Interestingly, while SF295 wild-type tumors showed significant growth advantage in SCIDhgf-Tg mice compared to that in SCID mice (FIG. 4A), a selected SF295SQ1 subclone which became more HGF-autocrine dominant after tumor passage (FIG. 2C) displayed the same growth in both models (FIG. 4B, FIG. 6C), further supporting the endogeneous HGF production influences the MET dependency in HGF-autocrine GBM tumors. The data also shows that HGF-autocrine status predicts HGF-dependent tumor growth susceptibility to MET inhibitors and hence may be useful as a predictive marker for targeting HGF autocrine GBM with MET inhibitors.

### Example 4

### Serum HGF Level Indicates Therapeutic Efficacy in HGF-Autocrine GBM Xenocraft Models

Because HGF is secreted by autocrine tumor cells into the circulation, a reduction of tumor size should result in a decrease of HGF production. The inventors asked if the serum HGF level can serve as a biomarker of therapeutic response. Using ELISA, the inventors determined the human HGF levels in the serum of the HGF-autocrine tumor-bearing mice from the in vivo MET drug efficacy study (FIGS. 6A-6E). SCID mice in the SGX523-alone and erlotinib-combination groups had much smaller tumors (FIGS. 6A-6C) accompanied by significantly lower serum HGF levels in the SCID mice (Table 1). It was expected that HGF expression in SCIDhgf-Tg mice would be much higher than SCID mice due to the expression of human HGF transgene. However, the strong HGF-paracrine stimulation in this mouse model did not influence the inhibition by SGX523 either alone or in combination with erlotinib (FIG. 6A-6C, SCID mice vs. SCIDhgf-Tg mice), suggesting that the HGF-autocrine status is sufficient to maintain activation of the MET signaling pathway and is the key determinant of sensitivity to MET inhibition.

TABLE 1
Serum HGF concentration in GBM in vivo models (pg/ml)

| | U87M2 | | SF295 SQ1 | | U118 | |
|---|---|---|---|---|---|---|
| Groups | SCIDhgf ¹ | SCID ² | SCIDhgf | SCID | SCIDhgf | SCID |
| Vehicle | 9703 ± 2989 | 1595 ± 305 | 4882 ± 2725 | 565 ± 162 | 5313 ± 487 | 513 ± 487 |
| SGX523 60 mg | 6330 ± 2240 | 152 ± 85 ⁴ | 4214 ± 1084 | 262 ± 138 ⁴ | 4149 ± 1264 | 91 ± 85 ^{5, 6} |
| Erlotinib 150 mg | 8233 ± 3311 | 1277 ± 679 | 4000 ± 1132 | 858 ± 670 | 2940 ± 746 | 348.7 ± 172 |
| Combination ³ | 12341 ± 2293 | 3.95 ± 5.59 ^{4, 6} | 4900 ± 2327 | 69 ± 65 ⁴ | 11647 ± 7942 | 93 ± 28 ⁶ |

At the end of the efficacy study shown in FIG. 6A, serum was collected from each group of mice and kept at −80° C. until use. To measure HGF expression, 50 μl of serum was used for an ELISA test. At least five samples were tested, and data represent mean ± SD. Student's t test was used for statistic analysis.
¹ SCIDhgf-Tg mice express high levels of human HGF.
² SCID mice do not express human HGF and the results reflect treatment efficacy.
³ “Combination” refers to 60 mg/kg SGX523 plus 150 mg/kg erlotinib.
⁴ One-tailed, p < 0.001.
⁵ p < 0.05.
⁶ Due to the toxicity observed in the Erlotinib and Combination groups, some mice were not available for blood withdrawal and fewer than 5 mice were tested for HGF level.

### Example 5

### A Combination of EGFR and c-Met Inhibitors Enhances Efficacy Against GBM

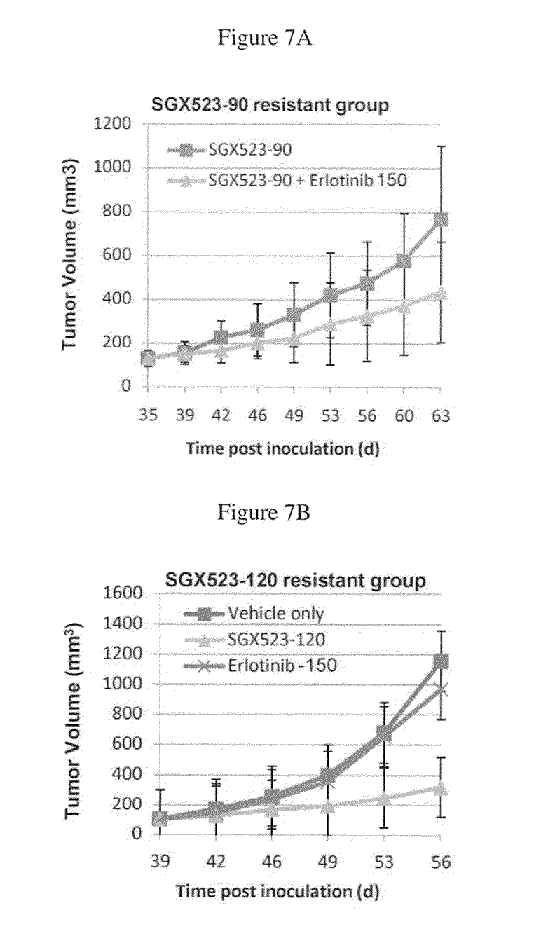

FIGS. 6A-6E also show that different GBM models may prefer either MET or EGFR inhibitor treatment alone (e.g., U87M2, U118, and SF295SQ1 are more sensitive to SGX523, while DBM2 is somewhat sensitive to erlotinib). Yet, U251M2 tumors, which tolerate either SGX523 or erlotinib alone, are significantly more sensitive to the combination even in SCID mice (FIG. 6D, p<0.05), where there is no HGF paracrine activation, nor do tumors have basal p-MET. While the exact mechanism is unknown, the inventors have previously observed similar results with other tumor xenograft models showing the combination of erlotinib and SGX523 inhibited tumor growth better than either drug alone in either SCIDhgf or SCID mice (24). Although SGX523 induces U87M2 tumor regression in the first week after treatment started (FIG. 6A), tumors start to grow back after 2 weeks in spite of continuous dosing with SGX523 (FIG. 7A-7B), implying selection of a rescue pathway. Because EGFR activation is often linked to MET signaling (15, 25, 26), the inventors tested whether erlotinib can enhance the effect of MET inhibition. The inventors showed that a combination of SGX523 and erlotinib can prolong the inhibition of U87M2 tumor growth that respond to SGX523 treatment (FIG. 7A, p<0.05), while erlotinib alone failed (FIG. 7B). Collectively, the data support the value of developing a combination of MET and EGFR inhibitors in targeting GBM.

### Example 6

### 7gain^{MET} in GBM Fails to Respond to MET Inhibition In Vivo

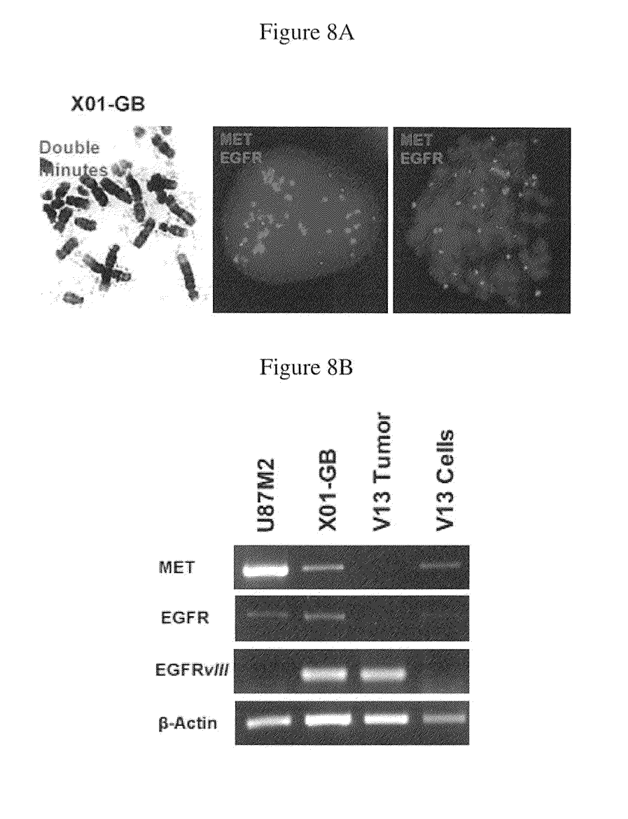

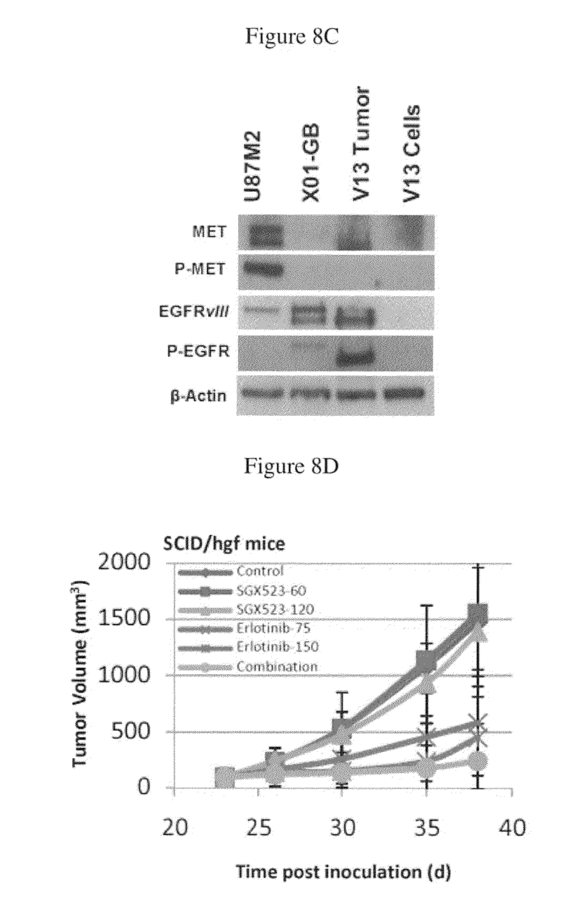

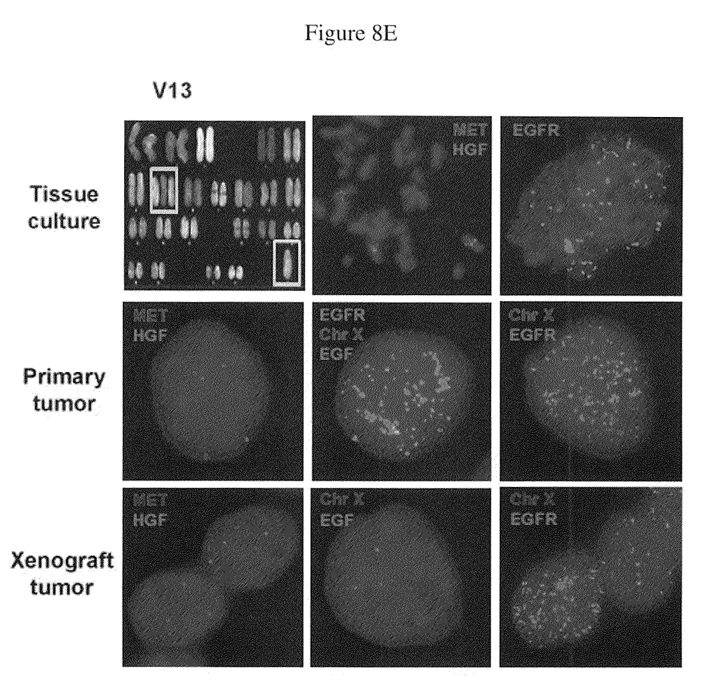

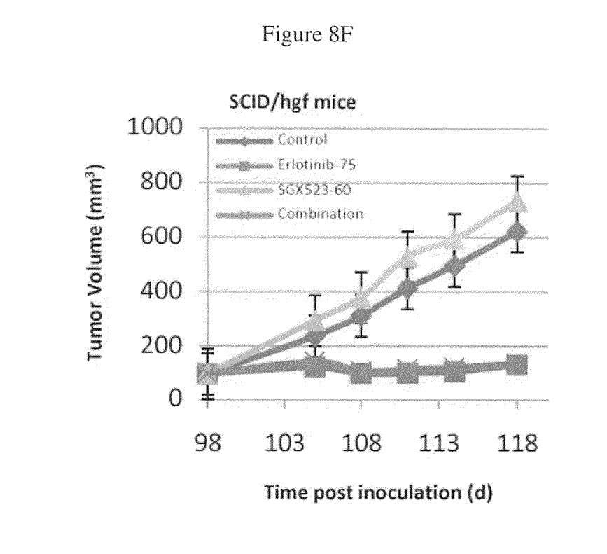

Recent studies have shown that approximately 88% GBM patients bear tumors having an altered RTK/RAS/PI3K pathway resulting from either EGFR (45%) or MET (4%) amplification (11, 12). To determine whether amplified MET or EGFR predicts the sensitivity to their specific inhibitors, the inventors examined GBM stem cell line X01-GB (27) for in vivo responsiveness to either MET or EGFR inhibition. Through chromosome analysis and fluorescence in situ hybridization (FISH) the inventors found that X01-GB had a ploidy level range from 4n-6n while harboring over 100 copies of EGFR and 7-10 copies of chromosome 7 and MET. The amplification of EGFR (EGFR^{amp}) is in the form of double minutes (dmin) and that of MET is in the form of gain of chromosome 7 (7gain^{MET}, FIG. 8A).

Consequently, the activation levels of the two receptors are very different. For example, X01-GB cells express high levels of EGFR and the mutant derivative EGFRvIII at both the transcriptional level (FIG. 8B) and protein level, with p-EGFRvIII (FIG. 8C). The expression of MET is quite low and there is no detectable p-MET, indicating a highly activated EGFR pathway while MET is inactive. Moreover, the X01-GB tumors do not respond to the MET inhibitor SGX523 but are very sensitive to erlotinib at 75 mg/kg (FIG. 8D). Western blot with tumor lysates consistently showed low MET without detectable p-MET, but strong EGFR activation that p-EGFR was significantly reduced by erlotinib or combination. HGF expression was not found either by Western blot with X01-GB tumors gown in SCID mice. These results suggest that while EGFR^{amp} might be used as a predictive marker for EGFR inhibitor sensitivity, 7gain^{MET} does not serve as an indicator of sensitivity to MET inhibition.

The inventors also tested a GBM patient specimen, V13, with similar cytogenetic analyses to identify the 7gain gain^{MET} and EGFR^{amp}. In order to gain as much chromosomal information as the inventors could, the inventors not only performed FISH on the V13 frozen primary tumor (FIG. 8E, middle panel), the inventors also established a tumor cell line from the primary tumor for more extensive analysis. The inventors were able to perform spectral karyotyping (SKY) and FISH on the V13 cell line while also performing FISH on the V13 primary and xenograft tumors to show consistency in the V13 samples. The inventors observed that the V13 primary tumor displayed 3 copies of HGF and MET, which are both located on chromosome 7, and amplification of EGFR (50-100 copies) (FIG. 8E). The SKY and FISH analysis of the V13 cell line clearly showed a gain of chromosome 7 and 10-100 dmin of EGFR (FIG. 8E), which is similar to the V13 primary tumor, xenograft tumor and sample X01-GB. A detailed comparison of the V13 cell line, the primary tumor, and the xenograft tumor by FISH analysis is summarized in Table 2.

TABLE 2
FISH analysis of MET and EGFR in V13 samples

| | Probe | | | | | | | | | |
|---|---|---|---|---|---|---|---|---|---|---|
| | HGF\MET | | EGF\Chr. X | | | | | | | |
| | (n = 200) | | (n = 200) | | EGFR (n = 200) | | | | | |
| No. of Signals | 2\2 | 3\3 | 2\1 | 2\2 | 2 | 3 | 10-29 | 30+ | 50+ | 100+ |
| V13 cells p4* | 78.0% | 22.0% | 37.0% | 63.0% | 76.5% | 15.0% | 2.0% | 4.0% | 2.0% | 0.5% |
| V13 primary | 12.5% | 87.5% | 4.0% | 96.0% | 4.0% | — | — | — | 96.0% | |
| tumor V13 | | | | | | | | | | |
| xenograft | — | 100.0% | — | 100.0% | — | — | — | 43.0% | 53.5% | 3.5% |
| tumor P0* | | | | | | | | | | |

*“p4” refers to the 4th passage after the cells were isolated from the primary tumor.
**“P0” refers to the first tumor transplant from a patient to a mouse model.

### Example 7

The inventors examined MET and EGFR expression levels in the V13 cell line and xenograft tumors, because V13 showed a cytogenetic profile similar to that of X01-GB which also had 7gain gain^{MET} and a very high EGFRvIII amplification. Moreover, the V13 xenograft tumors showed a very similar pattern to that of X01-GB, i.e., low MET expression without MET phosphorylation but a high level of EGFRvIII expression and phosphorylation, indicating activation of the EGFR signaling pathway (FIGS. 8B and 8C). The inventors did not detect MET or EGFR signaling in the V13 cell line likely because the majority of cells isolated from the primary tumor (78%) appear normal (Table 2). The V13 xenograft tumor was transplanted into SCIDhgf mice at an early passage number (p2) for an in vivo efficacy study in response to SGX523 or erlotinib, and it again showed identical results to X01-GB (FIG. 8F). V13 tumors are extremely sensitive to EGFR inhibitor where Erlotinib alone at 75 mg/kg induced tumor regression, but V13 showed no response to the MET inhibitor, SGX523. The combination of both inhibitors did not seem to enhance the efficacy, possibly due to the strong inhibitory effect of Erlotinib (FIG. 8F). Western blot with V13 tumor lysates showed similar results to X01-GB tumors—lack of MET and p-MET expression but with strong EGFR activation that p-EGFR expression can be significantly reduced by Erlotinib or combination treatment. Again, HGF expression was not found with V13 tumors grown in SCID mice. The inventors' data show that the lack of association between 7gain^{MET} and MET activity also holds for an ex vivo GBM and is not unique to a long term cell line.

### Example 8

### Aberrant Expression of HGF, MET and EGFR in Human GBM

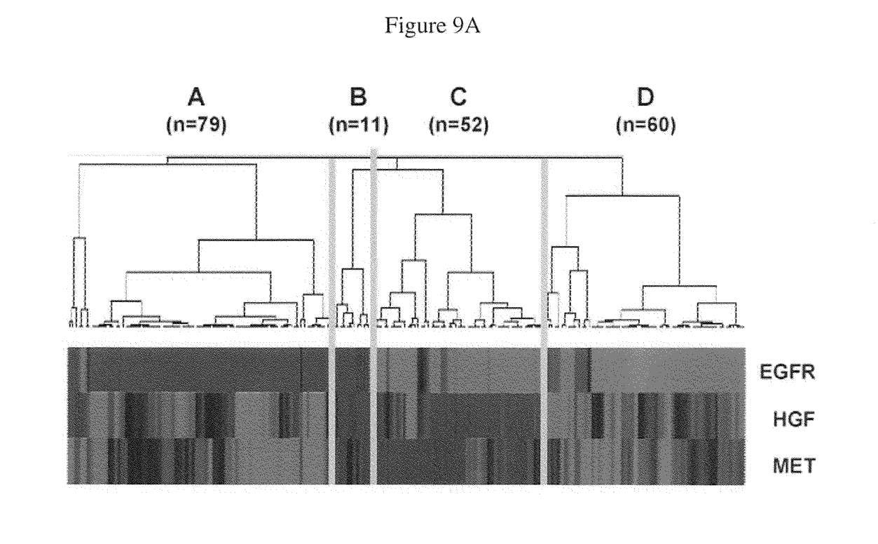

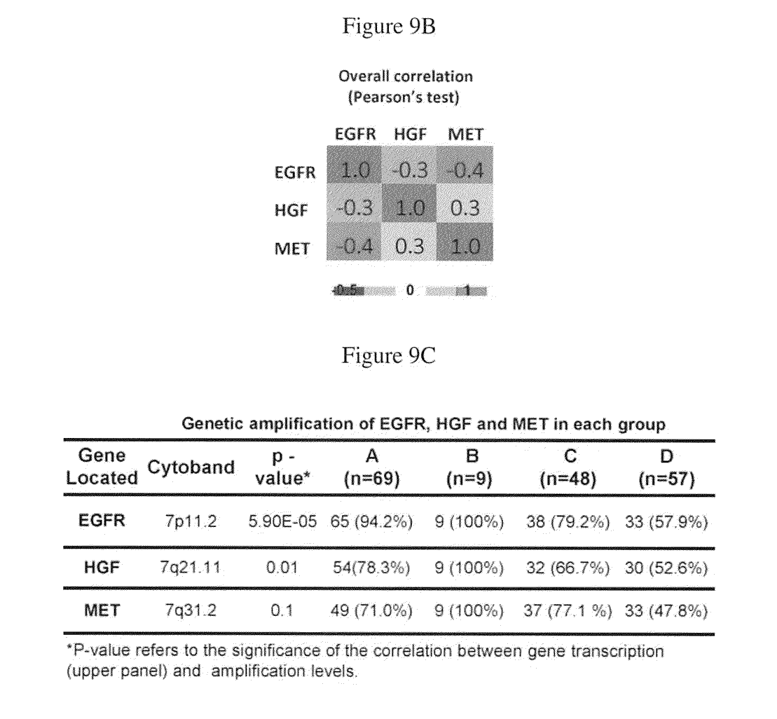

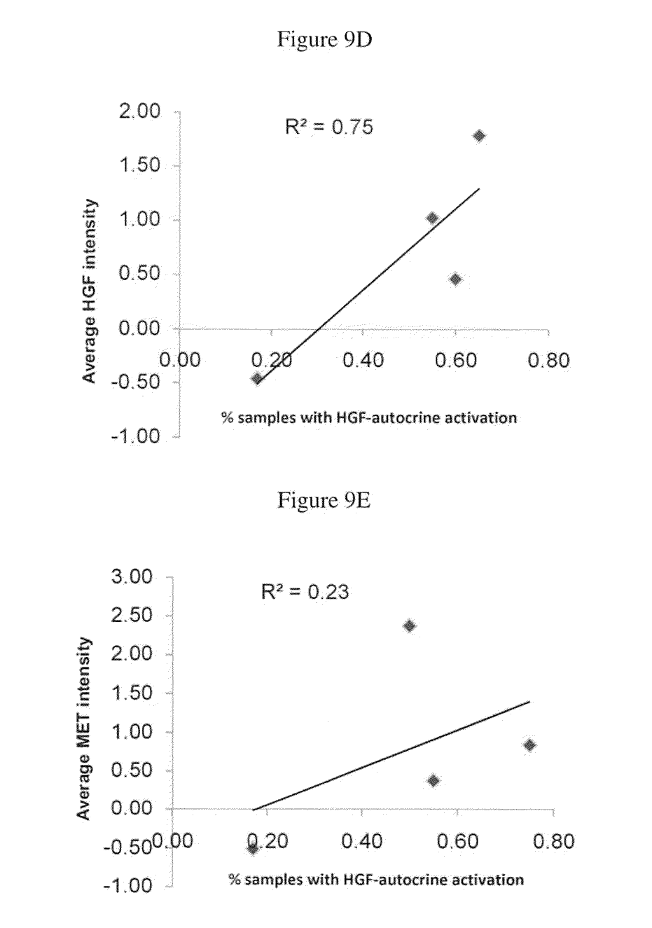

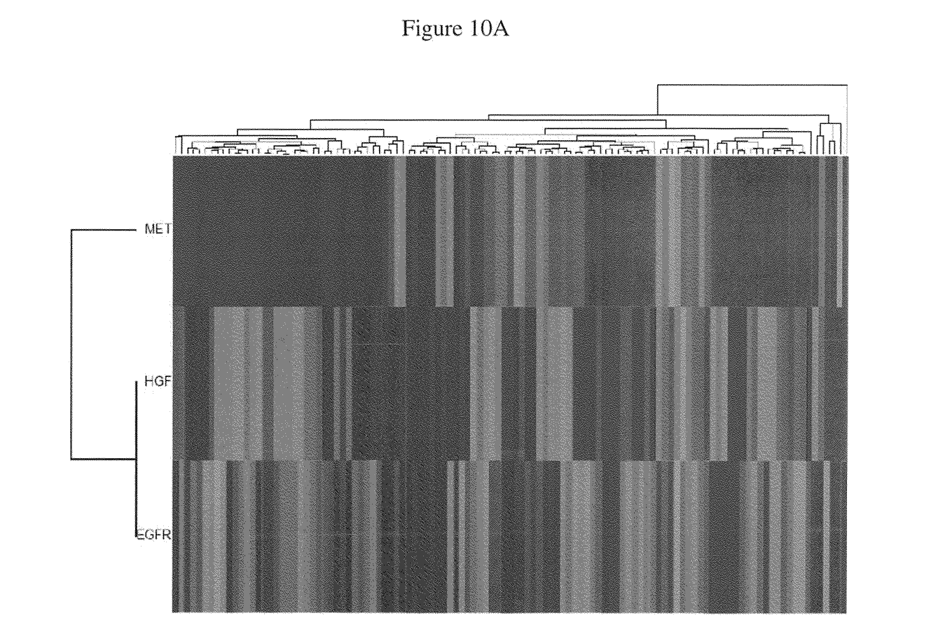

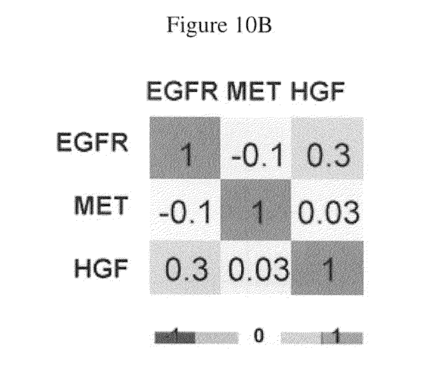

To estimate the frequency of HGF-autocrine, -paracrine, and 7gain^{MET} with EGFR^{amp} in GBM patients, the inventors analyzed the relationship of 202 GBM patients in silico assayed by the TCGA Network for the expression of EGFR, HGF and MET. A self organizing heat map displaying HGF, EGFR and MET genes, segregated the 202 GBM patients into 4 groups (FIG. 9A). A majority of samples (Group A, n=79) displayed high expression of EGFR and low expression of MET and HGF. However a significant number of samples (Group C, n=52), showed low expression of EGFR and high expression of HGF and MET. The remaining samples showed either low (Group D, n=60), or high (Group B, n=11) expression of these three genes. While 90 (45%) cases were over-expressing EGFR, 63 (31.2%) cases showed over-expression of both HGF and MET suggesting the possibility of autocrine HGF signaling. Interestingly, when looking into the individual cases, EGFR expression is frequently associated with low MET expression (Group A), whereas patients with high MET expression showed low EGFR (Group C). Thus, negative regulation may occur between the two pathways as indicated by the similarity matrix, which shows an overall negative correlation between the MET, and EGFR (n=202, Pearson's p=−0.4, FIG. 9B). These analyses suggest that the suppression of one pathway may result in an activation of another and might also explain why the combination of SGX523 and Erlotinib can further inhibit U87M2 tumor growth after they became resistant to SGX523. For comparison the inventors do not see this HGF-MET negative correlation with EGFR in melanoma patients samples (FIGS. 10A & 10B), suggesting this effect might be organ specific.

The inventors also estimated the percentage of patients with both EGFRamp and 7gain^{MET} as found with X01 GB and V13, where only EGFR is activated. The inventors used CGH data to analyze EGFR, HGF and MET amplification (FIG. 9C) and correlated the results to the transcriptional data (FIG. 9A). As previously reported, along with EGFR (94%), MET (78%), and HGF (71%), are frequently amplified, consistent with a high percentage of 7gain^{MET} (Table 3) (28).

TABLE 3
Genetic abberency of EGFR, HGF and MET with CGH analysis

| | Number of samples | | | |
|---|---|---|---|---|
| | with genetic | Type of aberration | | |
| Genes | aberrancy | Del. | Ampl. | Unch. |
| EGFR | 147 (80.3%) | 2 (1%) | 145 (79.2%) | 36 (19.6%) |
| HGF | 129 (70.4%) | 4 (2.1%) | 125 (68.3%) | 54 (29.5%) |
| MET | 129 (70.4%) | 1 (0.5%) | 128 (69.9%) | 54 (29.5%) |

However, when the inventors characterized Group A for levels of transcription (FIG. 9A), the MET and HGF genes in Group A were not highly expressed (FIG. 9A). By contrast EGFR amplification observed in group A (94%) are associated with high EGFR expression, showing a pattern similar to X01 GB and V13. These data suggested that amplification observed in EGFR gene (FIG. 9C) are significantly correlated with the gene expression of the 4 groups observed at transcriptional level (FIG. 9A, p=5.9E-5). However, no significant correlation was found for the MET gene (p=0.1), supporting the inventors' results (FIG. 8A-8D) that amplification occurring with the EGFR gene may predict an activation of EGFR pathway, whereas amplified MET may not. In this case, the majority of patients in amplified MET in group A are likely to be 7gain^{MET}. Interestingly, the inventors show here the copy number of HGF has better correlation to HGF overexpression (p=0.01) compared to MET, suggesting that HGF might be a better marker than MET.

### Example 9

Preclinical studies have shown that targeting MET signaling can have potent anti-tumor effects, including against GBM (29-32). MET inhibitors are currently in clinical trials against several cancers (17, 18). While AMG102, a neutralizing antibody against HGF failed in clinical trials against recurrent GBM (33), XL184, a small molecule targeting both MET and VEGF showed promising efficacy in phase II clinical trials (ASCO 2011). Therefore, it is important to understand the mechanisms leading to HGF/MET dependency and to identify the patient subgroups most likely to benefit from MET therapeutics. Unlike HER2/neu and Herceptin—where HER2/neu expression levels correlate with outcome in breast cancer, and a single monoclonal antibody against HER2/neu is efficacious—The inventors didn't see that MET expression linearly correlate with MET activation (FIG. 2C). Although MET expression is high in GBM patients (Koochekpour et al., 1997) and is used as a “mesenchymal marker” to indicate a more invasive GBM phenotype (12, 28), it may not be a good biomarker to predict the sensitivity to MET inhibition.

Efforts have been made to test p-MET levels with paraffin-embedded tissues, but still in need of success. Here, the inventors show that HGF-autocrine GBM tumors have constitutively activated MET in association with HGF expression levels, suggesting the use of HGF-autocrine activation as a biomarker to identify GBM patients potentially can be benefit from MET inhibitors. HGF is a circulating protein that can be easily measured by ELISA in serum or cerebrospinal fluid (CSF). Serum HGF levels correlate with GBM prognosis (34). More recently, HGF levels in the CSF of patients with GBM (847±155 pg/ml) was found to be significantly higher than in patients with meningioma (430±28 pg/ml) or in the healthy population (204±28 pg/ml). High HGF levels in patients with GBM are associated with higher mortality and recurrence rates (35).

Because high MET expression is found to be associated with high grade tumors (www.vai.org/met) and is under consideration as a biomarker, the inventors compared the use of HGF and MET as potential biomarker to predict HGF-autocrine loop signaling (Table 4, 5, FIG. 9D, 11E). Based on log2 signal intensity, 202 GBM samples were ranked from the highest to the lowest value of HGF and MET and the average signal intensity for each individual gene was calculated and presented in increments of 10% (n=20). The signal intensity significantly dropped after the top 30% (average HGF intensity=−0.46; average MET intensity=−0.51), therefore the first 60 cases (Top 30%) were considered to have “high” HGF or MET to analyze how well “higher” HGF samples are coincidental with “higher” MET expression, or vice versa, all the samples were sorted according to MET or HGF expression (Table 4 and 5).

TABLE 4
HGF-autocrine activation predicted with HGF expression level

| | | | | % of samples |
|---|---|---|---|---|
| | | | Numbers of | with HGF- |
| HGF | Number of | Average | samples with | autocrine |
| Expres- | samples | HGF | MET over- | activation |
| sion | (n1) | intensity | expression (n2) | (n2/n1) |
| Top 10% | 20 | 1.79 | 13 | 0.65 |
| 11-20% | 20 | 1.03 | 11 | 0.55 |
| 21-30% | 20 | 0.47 | 12 | 0.60 |
| 31-100% | 142 | −0.46 | 24 | 0.17 |

Using HGF expression as a marker to define the samples with HGF-autocrine activation. All samples were sorted and subdivided into 4 groups based upon the HGF signal intensity. The numbers of samples that have high MET from each group was identified; the frequency of samples with HGF-autocrine activation was calculated accordingly.

TABLE 5
HGF-autocrine activation predicted with MET expression level

| | | | | % of samples |
|---|---|---|---|---|
| | | | Numbers of | with HGF- |
| MET | Number of | Average | samples with | autocrine |
| Expres- | samples | MET | HGF over- | activation |
| sion | (n1) | intensity | expression (n2) | (n2/n1) |
| Top 10% | 20 | 2.37 | 10 | 0.50 |
| 11-20% | 20 | 0.84 | 15 | 0.75 |
| 21-30% | 20 | 0.38 | 11 | 0.55 |
| 31-100% | 142 | −0.51 | 24 | 0.17 |

Use MET expression as a marker to define the samples with HGF-autocrine activation. All samples were sorted and subdivided into 4 groups based upon the MET signal intensity. The numbers of samples that have high HGF from each group was identified; the frequency of samples with HGF-autocrine activation was calculated accordingly.

The results showed that among 20 samples with highest HGF expression (average HGF intensity=1.79), 13 samples were found also with MET over-expression. As the level of HGF decreased (11-20%, average HGF intensity=1.03), the samples with high MET also become less (FIGS. 4A and 4B). When the inventors used MET over-expression level as a marker, however, the identification of HGF over-expression was less informative. Among 20 samples with highest MET expression (average MET intensity=2.37), 10 cases showed “high” HGF, but as the level of MET expression become (11-20%, average MET intensity=0.84), 15 samples were identified with high HGF (Table 6). Compared with MET, the average HGF intensity showed better correlation with samples that could potentially be HGF-autocrine signaling (R2=0.75 vs. 0.25, FIG. 9D vs. 9E). The data suggest the use of HGF as a bio-marker will largely enhance the specificity in identifying samples with active MET pathway.

The inventors' data also showed that the serum HGF level is correlated with the therapeutic efficacy of SGX523 in HGF-autocrine GBMs xenograft models (Table 1), suggesting that circulating HGF may be a useful prognostic and therapeutic marker for GBM patients. In the SCIDhgf-Tg mouse preclinical model, the inventors observed that HGF-paracrine activation can promote GBM tumor growth (DBM2, FIGS. 8A-8F). However the activity appeared weaker than other cancer models tested in this system (24, 36), and barely showed any response to MET inhibitors pointing out the unusual differences in sensitivity between autocrine and paracrine activation. In other cancer types, MET amplification is involved in non-small cell lung cancer (NSCLC) patients resistant to EGFR inhibitors (25). Interestingly, all these cases were found to have increased expression of HGF in tumor sections (9), suggesting HGF can serve as a therapeutic marker for MET sensitivity. Autocrine HGF indication can be detected by IHC and in serum (or CSF) but is not so straight forward. Perhaps the level in body fluids can be diagnostic, but distinguishing paracrine- or HGF-autocrine by IHC is more difficult.

MET amplification is sensitive to MET inhibitor in gastric cancers (26). Because MET amplification occurs in 4% of GBM patients (11), the inventors tested whether GBM tumors having MET amplification are sensitive to MET inhibitors. Stem cells and xenograft tumor models (28, 37) have high clinical relevance. The inventors therefore tested X01-GB, a GBM stem cell line, and V13, a GBM xenograft tumor line derived from a GBM patient, for their response to MET and EGFR inhibitors. Both models have a 7gain^{MET} (3-10 copies) and EGFR^{amp} with 30-100 dmin. Surprisingly, unlike the gastric cancer line MKN45 and the NSCLC line H820, where MET amplification is always accompanied by constitutively activated MET (25, 26), neither V13 nor X01-GB displayed strong MET expression or detectable p-MET, but both displayed strong EGFRvIII expression and p-EGFR. Consistently, in vivo, these tumors did not respond to SGX523, but did respond to Erlotinib. Thus, a high level of EGFR^{amp} does predict sensitivity to EGFR inhibition in GBM but unless the tumor is HGF-autocrine, 7gain^{MET} may not to be a good predictor for MET sensitivity.

The inventors questioned whether a different profile of MET amplification would result in different MET activity. In both MKN45 and H820, MET signals were located on chromosome 7 and in areas distinct from the endogenous gene locus (25, 26), while in V13 and X01-GB, MET signals only come from chromosome 7. Thus, the constitutive activation of MET may be more associated with the aberrant MET locus. In fact, the EGFR amplification in V13 and X01-GB occur as extrachromosomal dmin and are highly associated with EGFR activation and sensitivity to EGFR inhibition. The inventors also examined the GBM cell lines tested in in vivo with FISH analysis (Table 6).

TABLE 6
FISH analysis of MET and EGFR in additional GBM cell lines ^{a}

| | Ploidy | No. of MET signals | | No. of EGFR signals | | |
|---|---|---|---|---|---|---|
| Cell lines | level | (% ofcells) | | (% ofcells) | | Chr. 7 ^{b} |
| U87M2 | 2n | 2 | (88%) | 2 | (88%) | normal |
| | 4n | 4 | (12%) | 4 | (12%) | |
| SF295SQ1 | 3n | 5 | (52%) | 5 | (52%) | gain |
| | 6n | 10 | (48%) | 10 | (48%) | |
| U118 | 4n | 4 (15%), 5 (66%), 6 (19%) | | 5 (15%), 6 (85%) | | gain |
| DBM2 | 3n | 4 | (100%) | 4 | (100%) | gain |

| U251M2 | 4n | 3 (28%), 4 (72%) | | 5 (28%), 6 (72%) | EGFR gain |
|---|---|---|---|---|---|
| X01-GB | 4-6n | 7-10 | (100%) | 8-10 (8%), 100+ ^{c} (92%) | gain |
| V13 xenograft | 2n | 3 | (100%) | 30+ ^{c} (43%), 50+ ^{c} (57%) | gain |
| tumor p0 | | | | | |

^{a} FISH probes used for MET and EGFR analysis were the same probes as described in Table 3.
^{b} Numerical change of Chr. 7 in relation to the cell line's ploidy level.
^{c} Signals shown as double minutes.

The inventors found that the gain of MET occurred frequently in these GBM cell lines. This is consistent to the inventors' in silico analysis with the TCGA database (FIGS. 8A-10A). SF295SQ1, U118, and DBM2 all have gain of chromosome 7, they can be either sensitive or insensitive to SGX523 (FIGS. 6A-6E). U87M2 shows sensitivity to SGX523 but does not have chromosome 7 gain, showing that even when not amplified, HGF autocrine tumors may still be sensitive to MET inhibitors. Thus, it is worthwhile to accurately evaluate GBM patient samples. In particular, the inventors suggest distinguishing 7gain^{MET} from MET^{amp}), as they may indicate different cellular activities. While interphase FISH analysis of frozen tumor tissues can only give us information on the number of gene copies, a more informative technique such as metaphase FISH and chromosome analysis that uses isolated tumor cells to identify the location of MET amplification or gain is needed.

Taken together, the inventors' study investigated HGF-autocrine and HGF-paracrine status, as well as MET and EGFR amplification as molecular determinants of HGF/MET dependency. The inventors found HGF-autocrine status can indicate MET activity and predict sensitivity to MET inhibitors. Because MET inhibitors are in clinical trials against GBM, it is worthwhile to expand these findings by further evaluating the use of HGF-autocrine status as a therapeutic marker for identifying patient subgroups for MET treatment. The inventors also found that a combination of MET and EGFR inhibitors inhibit the HGF-paracrine GBM models the inventors have tested, supporting the value of such a combination as a preferred strategy for treating malignant GBM. Since brain tumors are extremely heterogeneous, more information on how the genetic background and overlapping signaling networks influence tumor growth is needed to uncover the full potential of using novel combinations of reagents to treat malignant glioma.

### REFERENCES

1. Birchmeier C, Birchmeier W, Gherardi E, Vande Woude G F. Met, metastasis, motility and more. Nat Rev Mol Cell Biol 2003; 4(12):915-25.
2. Jeffers M, Rong S, Vande Woude G F. Enhanced tumorigenicity and invasion-metastasis by hepatocyte growth factor/scatter factor-met signalling in human cells concomitant with induction of the urokinase proteolysis network. Mol Cell Biol 1996; 16(3):1115-25.
3. Boccaccio C, Comoglio P M. Invasive growth: a MET-driven genetic programme for cancer and stem cells. Nature reviews 2006; 6(8):637-45.
4. Rao J S. Molecular mechanisms of glioma invasiveness: the role of proteases. Nature reviews 2003; 3(7):489-501.
5. Holland E C, Celestino J, Dai C, Schaefer L, Sawaya R E, Fuller G N. Combined activation of Ras and Akt in neural progenitors induces glioblastoma formation in mice. Nature genetics 2000; 25(1):55-7.
6. Holland E C. Gliomagenesis: genetic alterations and mouse models. Nat Rev Genet. 2001; 2(2):120-9.
7. Tsuda M, Davis I J, Argani P, et al. TFE3 fusions activate MET signaling by transcriptional up-regulation, defining another class of tumors as candidates for therapeutic MET inhibition. Cancer research 2007; 67(3):919-29.
8. Jeffers M, Fiscella M, Webb C P, Anver M, Koochekpour S, Vande Woude G F. The mutationally activated Met receptor mediates motility and metastasis. Proceedings of the National Academy of Sciences of the United States of America 1998; 95(24):14417-22.
9. Turke A B, Zejnullahu K, Wu Y L, et al. Preexistence and clonal selection of MET amplification in EGFR mutant NSCLC. Cancer cell 2010; 17(1):77-88.
10. Koochekpour S, Jeffers M, Rulong S, et al. Met and hepatocyte growth factor/scatter factor expression in human gliomas. Cancer research 1997; 57(23):5391-8.
11. Network CGAR. Comprehensive genomic characterization defines human glioblastoma genes and core pathways. Nature 2008; 455(7216):1061-8.
12. Verhaak R G, Hoadley K A, Purdom E, et al. Integrated genomic analysis identifies clinically relevant subtypes of glioblastoma characterized by abnormalities in PDGFRA, IDH1, EGFR, and NFL Cancer cell 2010; 17(1):98-110.
13. Fischer J, Palmedo G, von Knobloch R, et al. Duplication and overexpression of the mutant allele of the MET proto-oncogene in multiple hereditary papillary renal cell tumours. Oncogene 1998; 17(6):733-9.
14. Reznik T E, Sang Y, Ma Y, et al. Transcription-dependent epidermal growth factor receptor activation by hepatocyte growth factor. Mol Cancer Res 2008; 6(1):139-50.
15. Huang P H, Mukasa A, Bonavia R, et al. Quantitative analysis of EGFRvIII cellular signaling networks reveals a combinatorial therapeutic strategy for glioblastoma. Proceedings of the National Academy of Sciences of the United States of America 2007; 104(31):12867-72.
16. Lal B, Goodwin C R, Sang Y, et al. EGFRvIII and c-Met pathway inhibitors synergize against PTEN-null/EGFRvIII+ glioblastoma xenografts. Molecular cancer therapeutics 2009; 8(7):1751-60.
17. Eder J P, Vande Woude G F, Boerner S A, LoRusso P M. Novel therapeutic inhibitors of the c-Met signaling pathway in cancer. Clin Cancer Res 2009; 15(7):2207-14.
18. Knudsen B S, Vande Woude G. Showering c-MET-dependent cancers with drugs. Curr Opin Genet Dev 2008; 18(1):87-96.
19. Van Meir E G, Hadjipanayis C G, Norden A D, Shu H K, Wen P Y, Olson J J. Exciting new advances in neuro-oncology: the avenue to a cure for malignant glioma. CA: a cancer journal for clinicians 2010; 60(3):166-93.
20. Xie Q, Thompson R, Hardy K, et al. A highly invasive human glioblastoma pre-clinical model for testing therapeutics. Journal of translational medicine 2008; 6:77.
21. Xie Q, Gao C F, Shinomiya N, et al. Geldanamycins exquisitely inhibit HGF/SF-mediated tumor cell invasion. Oncogene 2005; 24(23):3697-707.
22. Webb C P, Hose C D, Koochekpour S, et al. The geldanamycins are potent inhibitors of the hepatocyte growth factor/scatter factor-met-urokinase plasminogen activator-plasmin proteolytic network. Cancer research 2000; 60(2):342-9.
23. Zhang Q, Yu Y A, Wang E, et al. Eradication of solid human breast tumors in nude mice with an intravenously injected light-emitting oncolytic vaccinia virus. Cancer research 2007; 67(20):10038-46.
24. Zhang Y W, Staal B, Essenburg C, et al. MET kinase inhibitor SGX523 synergizes with epidermal growth factor receptor inhibitor erlotinib in a hepatocyte growth factor-dependent fashion to suppress carcinoma growth. Cancer research 2010; 70(17):6880-90.
25. Bean J, Brennan C, Shih J Y, et al. MET amplification occurs with or without T790M mutations in EGFR mutant lung tumors with acquired resistance to gefitinib or erlotinib. Proceedings of the National Academy of Sciences of the United States of America 2007; 104(52):20932-7.
26. Smolen G A, Sordella R, Muir B, et al. Amplification of MET may identify a subset of cancers with extreme sensitivity to the selective tyrosine kinase inhibitor PHA-665752. Proceedings of the National Academy of Sciences of the United States of America 2006; 103(7):2316-21.
27. Oka N, Soeda A, Inagaki A, et al. VEGF promotes tumorigenesis and angiogenesis of human glioblastoma stem cells. Biochemical and biophysical research communications 2007; 360(3):553-9.
28. Phillips H S, Kharbanda S, Chen R, et al. Molecular subclasses of high-grade glioma predict prognosis, delineate a pattern of disease progression, and resemble stages in neurogenesis. Cancer cell 2006; 9(3):157-73.
29. Kim K J, Wang L, Su Y C, et al. Systemic anti-hepatocyte growth factor monoclonal antibody therapy induces the regression of intracranial glioma xenografts. Clin Cancer Res 2006; 12(4):1292-8.
30. Martens T, Schmidt N O, Eckerich C, et al. A novel one-armed anti-c-Met antibody inhibits glioblastoma growth in vivo. Clin Cancer Res 2006; 12(20 Pt 1):6144-52.
31. Buchanan S G, Hendle J, Lee P S, et al. SGX523 is an exquisitely selective, ATP-competitive inhibitor of the MET receptor tyrosine kinase with antitumor activity in vivo. Molecular cancer therapeutics 2009; 8(12):3181-90.
32. Cao B, Su Y, Oskarsson M, et al. Neutralizing monoclonal antibodies to hepatocyte growth factor/scatter factor (HGF/SF) display antitumor activity in animal models. Proceedings of the National Academy of Sciences of the United States of America 2001; 98(13):7443-8.
33. Wen P Y, Schiff D, Cloughesy T F, et al. A phase II study evaluating the efficacy and safety of AMG 102 (rilotumumab) in patients with recurrent glioblastoma. Neuro-oncology; 13(4):437-46.
34. Arrieta O, Garcia E, Guevara P, et al. Hepatocyte growth factor is associated with poor prognosis of malignant gliomas and is a predictor for recurrence of meningioma. Cancer 2002; 94(12):3210-8.
35. Garcia-Navarrete R, Garcia E, Arrieta O, Sotelo J. Hepatocyte growth factor in cerebrospinal fluid is associated with mortality and recurrence of glioblastoma, and could be of prognostic value. Journal of neuro-oncology 2010; 97(3):347-51.
36. Zhang Y W, Su Y, Lanning N, et al. Enhanced growth of human met-expressing xenografts in a new strain of immunocompromised mice transgenic for human hepatocyte growth factor/scatter factor. Oncogene 2005; 24(1):101-6.
37. Lee J, Kotliarova S, Kotliarov Y, et al. Tumor stem cells derived from glioblastomas cultured in bFGF and EGF more closely mirror the phenotype and genotype of primary tumors than do serum-cultured cell lines. Cancer cell 2006; 9(5):391-403.
38. Cristina Basilico, Addolorata Arnesano, Maria Galluzzo, Paolo M. Comoglio, and Paolo Michieli. A High Affinity Hepatocyte Growth Factor-binding Site in the Immunoglobulin-like Region of Met. Journal Of Biological Chemistry 2008; 283(30):21267-21277.
39. Sok J C, Coppelli F M, Thomas S M, et al. Mutant epidermal growth factor receptor (EGFRvIII) contributes to head and neck cancer growth and resistance to EGFR targeting. Clin Cancer Res 2006; 12(17):5064-73.
40. Simon R, Lam A, Li M C, Ngan M, Menenzes S, Zhao Y. Analysis of gene expression data using BRB-ArrayTools. Cancer informatics 2007; 3:11-7.
41. Eisen M B, Spellman P T, Brown P O, Botstein D. Cluster analysis and display of genome-wide expression patterns. Proceedings of the National Academy of Sciences of the United States of America 1998; 95(25):14863-8.
   While the present technology have been illustrated and exemplified throughout the description and in the Examples, it is obvious to one of ordinary skill that many changes may be made in the details of the process of assembly without departing from the spirit and scope of this disclosure.

What is claimed is:

1. A method for determining the responsiveness of a Met-related cancer in a subject to treatment with a Met inhibitor, the method comprising:
in a subject having or suspected of having a Met-related cancer, detecting whether the Met-related cancer is HGF-autocrine;
wherein if the Met-related cancer is HGF-autocrine, determining that the Met-related cancer will be responsive to treatment with a Met inhibitor.

2. The method according to claim 1, wherein the detecting step further comprises:
(a) obtaining a biological sample from the subject;
(b) measuring the level of expression of HGF in the biological sample; and
(c) comparing the level of expression of HGF present in the biological sample to a reference sample,
wherein, if the level of expression of HGF in the biological sample is different from the level of expression of HGF in the reference sample, then the Met-related cancer is HGF-autocrine.

3. The method according to claim 2, wherein the obtaining the biological sample from the subject is selected from the group consisting of: obtaining a blood sample from the subject, obtaining a tumor tissue specimen from the subject, obtaining a cerebral spinal fluid (CSF) sample from the subject, or combinations thereof.

4. The method according to claim 3, wherein a tumor tissue specimen is obtained from the subject.

5. The method according to claim 4, wherein a tumor tissue biopsy or a surgically resected tumor tissue sample is obtained from the subject.

6. The method according to claim 2, the measuring step further comprises contacting the biological sample with an HGF-binding reagent under conditions that promote the binding of HGF with the HGF-binding reagent

7. The method according to claim 6, wherein the HGF-binding reagent is selected from the group consisting of: an anti-HGF antibody, a nucleic acid operable to hybridize to at least a portion of an HGF gene, an HGF binding protein, and combinations thereof.

8. The method according to claim 7, wherein the HGF-binding reagent is labeled with a detectable tag.

9. The method according to claim 8, wherein the detectable tag is selected from the group consisting of: a fluorophore, a radioligand, a chemilluminescence molecule, a conjugated enzyme, a peptide conjugate molecule, and combinations thereof.

10. The method according to claim 7, wherein an anti-HGF antibody is conjugated with a fluorophore.

11. The method according to claim 10, wherein the fluorophore is selected from the group consisting of: fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) and N,N,N′,N′-tetramethyl-6-carboxyrhodamine (TAMRA)

12. The method according to claim 7, wherein a radioligand is conjugated to at least one of an anti-HGF antibody, a nucleic acid operable to hybridize to at least a portion of a HGF gene, and a HGF binding protein.

13. The method according to claim 7, wherein a conjugated enzyme is selected from the group consisting of: horseradish peroxidase, glucose oxidase, and alkaline phosphatase.

14. The method according to claim 7, wherein a peptide conjugate molecule is selected from the group consisting of: avidin, streptavidin, biotin, hexa-His, glutathione S-transferase (GST), and FLAG.

15. The method according to claim 7, wherein the HGF-binding reagent is a nucleic acid operable to hybridize to at least a portion of an HGF gene and the nucleic acid comprises a nucleotide of SEQ ID NOs: 1 or 2.

16. The method according to claim 2, wherein the level of expression of HGF in the biological sample is measured by immunohistochemistry (NC), enzyme linked immunosorbant assay (ELISA), reverse transcription-polymerase chain reation (RT-PCR), microarray analysis, in-vivo molecular imaging, or combinations thereof.

17. The method according to claim 2, wherein the reference sample is selected from the group consisting of a tissue-matched healthy control, a tissue-matched sample of the subject prior to diagnosis of cancer, and a tissue-matched sample of the subject prior to treatment.

18. The method according to claim 1, wherein the Met-related cancer is glioblastomamultiforme.

19. The method of claim 1, further comprising administering a therapeutically effective amount of a Met inhibitor to treat the Met-related cancer in the subject if it is detected that the Met-related cancer is HGF-autocrine.

20. The method according to claim 1, wherein an HGF-binding reagent is administered directly to the subject to detect whether the Met-related cancer is HGF-autocrine.

21. A method for determining the responsiveness of a Met-related glioblastoma cancer in a subject to treatment with a Met inhibitor, the method comprising:
(a) obtaining a glioblastoma tumor tissue biopsy sample from the subject;
(b) measuring the level of expression of HGF in the biological sample; and
(c) comparing the level of expression of HGF present in the biopsy sample to a reference sample,
wherein if the level of expression of HGF in the biopsy sample is different from the level of expression of HGF in the reference sample, the subject is identified as having a glioblastoma cancer which is sensitive to treatment with a Met inhibitor.

22. A kit for determining the responsiveness of a Met-expressing tumor to Met inhibition, the kit comprising:
a container for collecting a biological sample from a subject and a HGF-binding reagent for detecting HGF in the biological sample.

23. The kit of claim 22, wherein the HGF-binding reagent is selected from the group consisting of: an anti-HGF antibody, a nucleic acid operable to hybridize to at least a portion of a HGF gene, a HGF binding protein, and combinations thereof.

24. The kit of claim 23, wherein the HGF-binding reagent further comprises a detectable tag.

25. The kit of claim 24, wherein the detectable tag is selected from the group consisting of: a fluorophore, a radioligand, a chemilluminescence molecule, a conjugated enzyme, a peptide conjugate molecule, and combinations thereof.

26. A method of treating glioblastomamultiforme (GBM) in a subject in need thereof, the method comprising:
administering a therapeutically effective dose of a Met inhibitor in combination with a therapeutically effective dose of a epithelial growth factor receptor (EGFR) inhibitor.

27. The method according to claim 26, wherein the Met inhibitor is administered prior to or subsequent to the administration of the EGFR inhibitor.

28. The method according to claim 26, wherein the Met inhibitor is administered concomitantly with the EGFR inhibitor.
